SEQUENCE LISTING (76 sequences)

SEQ ID NO: 1 - protein, 21 aa, synthetic construct
MWWRLWWLLLLLLLLWPMVWA

SEQ ID NO: 2 - protein, 7 aa, synthetic construct
SNTRVAP

SEQ ID NO: 3 - protein, 8 aa, synthetic construct
GPLGMLSQ

SEQ ID NO: 4 - protein, 7 aa, synthetic construct
RSSYYSL

SEQ ID NO: 5 - protein, 7 aa, synthetic construct
LSLITRL

SEQ ID NO: 6 - protein, 5 aa, synthetic construct
TRSAW

SEQ ID NO: 7 - DNA, 43 bp, synthetic construct
  source 1..43: mol_type = other DNA
caaagcaaccggtcgcacgtcatgagccaccatgtggtggagg

SEQ ID NO: 8 - DNA, 43 bp, synthetic construct
  source 1..43: mol_type = other DNA
gtcattggggaaacctgctcctaggggctcatggagccaccct

SEQ ID NO: 9 - DNA, 75 bp, synthetic construct
  source 1..75: mol_type = other DNA
caaagcaaccggtcgcacgtcatgagccaccatgggtaacgaagcgtcttacccgctgagtggtggaggctgtgg

SEQ ID NO: 10 - DNA, 43 bp, synthetic construct; identical to SEQ ID NO: 8
  source 1..43: mol_type = other DNA

SEQ ID NO: 11 - DNA, 40 bp, synthetic construct
  source 1..40: mol_type = other DNA
taattcaaagcaaccggtcgtagccaccatgtggtggagg

SEQ ID NO: 12 - DNA, 51 bp, synthetic construct
  source 1..51: mol_type = other DNA
agttcttctcccttgctcatgccgctcgagcctggagccaccctggtgttg

SEQ ID NO: 13 - DNA, 19 bp, synthetic construct
  source 1..19: mol_type = other DNA
gtctggacagcctctggga

SEQ ID NO: 14 - DNA, 20 bp, synthetic construct
  source 1..20: mol_type = other DNA
tggtggctaagagcaacacc

SEQ ID NO: 15 - protein, 5 aa, synthetic construct
  MOD_RES 1: note = P is conjugated to 7-methoxycoumarin (Mca).
  MOD_RES 4..5: note = Residues 4 and 5 are linked by 2,4-dinitrophenyl (Dnp).
  MOD_RES 5: note = R has an NH2 terminal modification.
PLGLR

SEQ ID NO: 16 - protein, 17 aa, synthetic construct
  MOD_RES 1: note = T is conjugated to 7-methoxycoumarin (Mca).
  MOD_RES 17: note = K is conjugated to 2,4-dinitrophenyl (Dnp).
TRSAWGRSGSNTRVAPK

SEQ ID NO: 17 - protein, 25 aa, synthetic construct
  MOD_RES 1: note = T is conjugated to 7-methoxycoumarin (Mca).
  MOD_RES 25: note = K is conjugated to 2,4-dinitrophenyl (Dnp).
TRSAWGRSGQPLGLVAKSNTRVAPK

SEQ ID NO: 18 - protein, 7 aa, synthetic construct
  MOD_RES 1: note = S is conjugated to fluorescein isothiocyanate (FITC) via 6-aminohexanoic acid (Ahx).
SNTRVAP

SEQ ID NO: 19 - protein, 12 aa, synthetic construct
YHWYGYTPQNVI

SEQ ID NO: 20 - protein, 8 aa, synthetic construct
WIFPWIQL

SEQ ID NO: 21 - protein, 12 aa, synthetic construct
WDLAWMFRLPVG

SEQ ID NO: 22 - protein, 36 aa, synthetic construct
  MOD_RES 1: note = A signal peptide from tissue plasminogen activator (tPA) is conjugated to residue 1.
  MOD_RES 36: note = An epidermal growth factor receptor (EGFR) targeting motif (F-EGFR-A) is conjugated to the last residue of the peptide.
GPLGMLSQLSLITRLGPLGMLSQTRSAWGPLGMLSQ

SEQ ID NO: 23 - protein, 36 aa, synthetic construct; identical to SEQ ID NO: 22
  MOD_RES 1: note = A signal peptide from tissue plasminogen activator (tPA) is conjugated to residue 1.
  MOD_RES 36: note = An epidermal growth factor receptor (EGFR) targeting motif (R-EGFR-A) is conjugated to the last residue of the peptide.

SEQ ID NO: 24 - protein, 40 aa, synthetic construct
  MOD_RES 1: note = A signal peptide from tissue plasminogen activator (tPA) is conjugated to residue 1.
  MOD_RES 40: note = An epidermal growth factor receptor (EGFR) targeting motif (R-EGFR-A) is conjugated to the last residue of the peptide.
GRSGGPLGMLSQLSLITRLGPLGMLSQTRSAWGPLGMLSQ

SEQ ID NO: 25 - protein, 49 aa, synthetic construct
  MOD_RES 1: note = A signal peptide from tissue plasminogen activator (tPA) is conjugated to residue 1.
  MOD_RES 49: note = An epidermal growth factor receptor (EGFR) targeting motif (R-EGFR-A) is conjugated to the last residue of the peptide.
GRSGGPLGMLSQLSLITRLGRSGGPLGMLSQTRSAWGRSGGPLGMLSQI

SEQ ID NO: 26 - protein, 60 aa, synthetic construct
  MOD_RES 1: note = A signal peptide from tissue plasminogen activator (tPA) is conjugated to residue 1.
  MOD_RES 60: note = An epidermal growth factor receptor (EGFR) targeting motif (R-EGFR-A) is conjugated to the last residue of the peptide.
GRSGGRSGGPLGMLSQLSLITRLGRSGGRSGGPLGMLSQTRSAWGRSGGRSGGPLGMLSQ

SEQ ID NO: 27 - protein, 48 aa, synthetic construct
  MOD_RES 1: note = A signal peptide from tissue plasminogen activator (tPA) is conjugated to residue 1.
  MOD_RES 48: note = An epidermal growth factor receptor (EGFR) targeting motif (R-EGFR-A) is conjugated to the last residue of the peptide.
GGGSGPLGMLSQLSLITRLGGGSGPLGMLSQTRSAWGGGSGPLGMLSQ

SEQ ID NO: 28 - protein, 44 aa, synthetic construct
  MOD_RES 1: note = A signal peptide from tissue plasminogen activator (tPA) is conjugated to residue 1.
  MOD_RES 44: note = An epidermal growth factor receptor (EGFR) targeting motif (R-EGFR-A) is conjugated to the last residue of the peptide.
GPLGMLSQLSLITRLGPLGMLSQGRSGTRSAWGRSGGPLGMLSQ

SEQ ID NO: 29 - protein, 44 aa, synthetic construct
  MOD_RES 1: note = A signal peptide from tissue plasminogen activator (tPA) is conjugated to residue 1.
  MOD_RES 44: note = An epidermal growth factor receptor (EGFR) targeting motif (R-EGFR-A) is conjugated to the last residue of the peptide.
GPLGMLSQLSLITRLGPLGMLSQGRSGTRSAWGPSGGPLGMLSQ

SEQ ID NO: 30 - protein, 48 aa, synthetic construct
  MOD_RES 1: note = A signal peptide from tissue plasminogen activator (tPA) is conjugated to residue 1.
  MOD_RES 48: note = An epidermal growth factor receptor (EGFR) targeting motif (R-EGFR-A) is conjugated to the last residue of the peptide.
GPLGMLSQLSLITRLGPLGMLSQGRSGTRSAWGRSGGPLGMLSQGRSG

SEQ ID NO: 31 - protein, 48 aa, synthetic construct; identical to SEQ ID NO: 30
  MOD_RES 1: note = A signal peptide from tissue plasminogen activator (tPA) is conjugated to residue 1.
  MOD_RES 48: note = An epidermal growth factor receptor (EGFR) targeting motif (F-EGFR-A) is conjugated to the last residue of the peptide.

SEQ ID NO: 32 - protein, 44 aa, synthetic construct
  MOD_RES 1: note = A signal peptide from tissue plasminogen activator (tPA) is conjugated to residue 1.
  MOD_RES 44: note = An epidermal growth factor receptor (EGFR) targeting motif (R-EGFR-A) is conjugated to the last residue of the peptide.
GPLGMLSQLSLITRLGPLGMLSQTRSAWGRSGGPLGMLSQGRSG

SEQ ID NO: 33 - protein, 44 aa, synthetic construct; identical to SEQ ID NO: 32
  MOD_RES 1: note = A signal peptide from tissue plasminogen activator (tPA) is conjugated to residue 1.
  MOD_RES 44: note = An epidermal growth factor receptor (EGFR) targeting motif (F-EGFR-A) is conjugated to the last residue of the peptide.

SEQ ID NO: 34 - protein, 40 aa, synthetic construct
  MOD_RES 1: note = A signal peptide from tissue plasminogen activator (tPA) is conjugated to residue 1.
  MOD_RES 40: note = An epidermal growth factor receptor (EGFR) targeting motif (R-EGFR-A) is conjugated to the last residue of the peptide.
GPLGMLSQLSLITRLGPLGMLSQTRSAWGRSGGPLGMLSQ

SEQ ID NO: 35 - protein, 40 aa, synthetic construct; identical to SEQ ID NO: 34
  MOD_RES 1: note = A signal peptide from tissue plasminogen activator (tPA) is conjugated to residue 1.
  MOD_RES 40: note = An epidermal growth factor receptor (EGFR) targeting motif (F-EGFR-A) is conjugated to the last residue of the peptide.

SEQ ID NO: 36 - protein, 52 aa, synthetic construct
  MOD_RES 1: note = A signal peptide from tissue plasminogen activator (tPA) is conjugated to residue 1.
  MOD_RES 52: note = An epidermal growth factor receptor (EGFR) targeting motif (R-EGFR-A) is conjugated to the last residue of the peptide.
GPLGMLSQLSLITRLGRSGGPLGMLSQGRSGTRSAWGRSGGPLGMLSQGRSG

SEQ ID NO: 37 - protein, 52 aa, synthetic construct; identical to SEQ ID NO: 36
  MOD_RES 1: note = A signal peptide from tissue plasminogen activator (tPA) is conjugated to residue 1.
  MOD_RES 52: note = An epidermal growth factor receptor (EGFR) targeting motif (F-EGFR-A) is conjugated to the last residue of the peptide.

SEQ ID NO: 38 - protein, 48 aa, synthetic construct
  MOD_RES 1: note = A signal peptide from tissue plasminogen activator (tPA) is conjugated to residue 1.
  MOD_RES 48: note = An epidermal growth factor receptor (EGFR) targeting motif (R-EGFR-A) is conjugated to the last residue of the peptide.
GPLGMLSQLSLITRLGRSGGPLGMLSQGRSGTRSAWGRSGGPLGMLSQ

SEQ ID NO: 39 - protein, 5 aa, synthetic construct
PRSLT

SEQ ID NO: 40 - protein, 6 aa, synthetic construct
PLGLVA

SEQ ID NO: 41 - protein, 8 aa, synthetic construct
QPLGLVAK

SEQ ID NO: 42 - protein, 36 aa, synthetic construct; identical to SEQ ID NO: 22
  MOD_RES 1: note = A Secrecon peptide (Sec) is conjugated to residue 1.
  MOD_RES 36: note = A tumor-targeting peptide GE11 (F-GE11) is conjugated to the last residue of the peptide.

SEQ ID NO: 43 - protein, 48 aa, synthetic construct
  MOD_RES 1: note = A Secrecon peptide (Sec) is conjugated to residue 1.
  MOD_RES 48: note = A tumor-targeting peptide GE11 (F-GE11) is conjugated to the last residue of the peptide.
GRSGGPLGMLSQLSLITRLGRSGGPLGMLSQTRSAWGRSGGPLGMLSQ

SEQ ID NO: 44 - protein, 68 aa, synthetic construct
  MOD_RES 1: note = A Secrecon peptide (Sec) is conjugated to residue 1.
  MOD_RES 68: note = A tumor-targeting peptide GE11 (F-GE11) is conjugated to the last residue of the peptide.
GRSGGRSGGPLGMLSQLSLITRLGRSGGRSGGRSGGPLGMLSQTRSAWGRSGGRSGGRSGGPLGMLSQ

SEQ ID NO: 45 - protein, 51 aa, synthetic construct
  MOD_RES 1: note = A Secrecon peptide (Sec) is conjugated to residue 1.
  MOD_RES 51: note = A tumor-targeting peptide GE11 (F-GE11) is conjugated to the last residue of the peptide.
GGGGSGPLGMLSQLSLITRLGGGGSGPLGMLSQTRSAWGGGGSGPLGMLSQ

SEQ ID NO: 46 - protein, 63 aa, synthetic construct
  MOD_RES 1: note = A Secrecon peptide (Sec) is conjugated to residue 1.
  MOD_RES 63: note = A tumor-targeting peptide GE11 (F-GE11) is conjugated to the last residue of the peptide.
GGSGGGSGGGPLGMLSQLSLITRLGGSGGGSGGGPLGMLSQTRSAWGGSGGGSGGGPLGMLSQ

SEQ ID NO: 47 - protein, 44 aa, synthetic construct; identical to SEQ ID NO: 28
  MOD_RES 1: note = A Secrecon peptide (Sec) is conjugated to residue 1.
  MOD_RES 44: note = A tumor-targeting peptide GE11 (F-GE11) is conjugated to the last residue of the peptide.

SEQ ID NO: 48 - protein, 48 aa, synthetic construct; identical to SEQ ID NO: 30
  MOD_RES 1: note = A Secrecon peptide (Sec) is conjugated to residue 1.
  MOD_RES 48: note = A tumor-targeting peptide GE11 (F-GE11) is conjugated to the last residue of the peptide.

SEQ ID NO: 49 - protein, 44 aa, synthetic construct; identical to SEQ ID NO: 32
  MOD_RES 1: note = A Secrecon peptide (Sec) is conjugated to residue 1.
  MOD_RES 44: note = A tumor-targeting peptide GE11 (F-GE11) is conjugated to the last residue of the peptide.

SEQ ID NO: 50 - protein, 40 aa, synthetic construct; identical to SEQ ID NO: 34
  MOD_RES 1: note = A Secrecon peptide (Sec) is conjugated to residue 1.
  MOD_RES 40: note = A tumor-targeting peptide GE11 (F-GE11) is conjugated to the last residue of the peptide.

SEQ ID NO: 51 - protein, 52 aa, synthetic construct
  MOD_RES 1: note = A Secrecon peptide (Sec) is conjugated to residue 1.
  MOD_RES 52: note = A tumor-targeting peptide GE11 (F-GE11) is conjugated to the last residue of the peptide.
GRSGGPLGMLSQLSLITRLGPLGMLSQGRSGTRSAWGRSGGPLGMLSQGRSG

SEQ ID NO: 52 - protein, 56 aa, synthetic construct
  MOD_RES 1: note = A Secrecon peptide (Sec) is conjugated to residue 1.
  MOD_RES 56: note = A tumor-targeting peptide GE11 (F-GE11) is conjugated to the last residue of the peptide.
GRSGGPLGMLSQLSLITRLGRSGGPLGMLSQGRSGTRSAWGRSGGPLGMLSQGRSG

SEQ ID NO: 53 - protein, 27 aa, synthetic construct
  MOD_RES 1: note = A Secrecon peptide (Sec) is conjugated to residue 1.
  MOD_RES 27: note = A tumor-targeting peptide GE11 (F-GE11) is conjugated to the last residue of the peptide.
PRSLTLSLITRLPRSLTTRSAWPRSLT

SEQ ID NO: 54 - protein, 39 aa, synthetic construct
  MOD_RES 1: note = A Secrecon peptide (Sec) is conjugated to residue 1.
  MOD_RES 39: note = A tumor-targeting peptide GE11 (F-GE11) is conjugated to the last residue of the peptide.
GRSGPRSLTLSLITRLGRSGPRSLTTRSAWGRSGPRSLT

SEQ ID NO: 55 - protein, 51 aa, synthetic construct
  MOD_RES 1: note = A Secrecon peptide (Sec) is conjugated to residue 1.
  MOD_RES 51: note = A tumor-targeting peptide GE11 (F-GE11) is conjugated to the last residue of the peptide.
GRSGGRSGPRSLTLSLITRLGRSGGRSGPRSLTTRSAWGRSGGRSGPRSLT

SEQ ID NO: 56 - protein, 36 aa, synthetic construct
  MOD_RES 1: note = A Secrecon peptide (Sec) is conjugated to residue 1.
  MOD_RES 36: note = A tumor-targeting peptide GE11 (F-GE11) is conjugated to the last residue of the peptide.
GPLGLVAQLSLITRLGPLGLVAQTRSAWGPLGLVAQ

SEQ ID NO: 57 - protein, 48 aa, synthetic construct
  MOD_RES 1: note = A Secrecon peptide (Sec) is conjugated to residue 1.
  MOD_RES 48: note = A tumor-targeting peptide GE11 (F-GE11) is conjugated to the last residue of the peptide.
GRSGGPLGLVAQLSLITRLGRSGGPLGLVAQTRSAWGRSGGPLGLVAQ

SEQ ID NO: 58 - protein, 45 aa, synthetic construct
  MOD_RES 1: note = A Secrecon peptide (Sec) is conjugated to residue 1.
  MOD_RES 45: note = A tumor-targeting peptide GE11 (F-GE11) is conjugated to the last residue of the peptide.
GRSGPLGLVAQLSLITRLGRSGPLGLVAQTRSAWGRSGPLGLVAQ

SEQ ID NO: 59 - protein, 60 aa, synthetic construct
  MOD_RES 1: note = A Secrecon peptide (Sec) is conjugated to residue 1.
  MOD_RES 60: note = A tumor-targeting peptide GE11 (F-GE11) is conjugated to the last residue of the peptide.
GRSGGRSGGPLGLVAQLSLITRLGRSGGRSGGPLGLVAQTRSAWGRSGGRSGGPLGLVAQ

SEQ ID NO: 60 - protein, 57 aa, synthetic construct
  MOD_RES 1: note = A Secrecon peptide (Sec) is conjugated to residue 1.
  MOD_RES 57: note = A tumor-targeting peptide GE11 (F-GE11) is conjugated to the last residue of the peptide.
GRSGGRSGPLGLVAQLSLITRLGRSGGRSGPLGLVAQTRSAWGRSGGRSGPLGLVAQ

SEQ ID NO: 61 - protein, 36 aa, synthetic construct
  MOD_RES 1: note = A Secrecon peptide (Sec) is conjugated to residue 1.
  MOD_RES 36: note = A tumor-targeting peptide GE11 (F-GE11) is conjugated to the last residue of the peptide.
QPLGLVAKLSLITRLQPLGLVAKTRSAWQPLGLVAK

SEQ ID NO: 62 - protein, 48 aa, synthetic construct
  MOD_RES 1: note = A Secrecon peptide (Sec) is conjugated to residue 1.
  MOD_RES 48: note = A tumor-targeting peptide GE11 (F-GE11) is conjugated to the last residue of the peptide.
GRSGQPLGLVAKLSLITRLGRSGQPLGLVAKTRSAWGRSGQPLGLVAK

SEQ ID NO: 63 - protein, 60 aa, synthetic construct
  MOD_RES 1: note = A Secrecon peptide (Sec) is conjugated to residue 1.
  MOD_RES 60: note = A tumor-targeting peptide GE11 (F-GE11) is conjugated to the last residue of the peptide.
GRSGGRSGQPLGLVAKLSLITRLGRSGGRSGQPLGLVAKTRSAWGRSGGRSGQPLGLVAK

SEQ ID NO: 64 - protein, 51 aa, synthetic construct
  MOD_RES 1: note = A Secrecon peptide (Sec) is conjugated to residue 1.
  MOD_RES 51: note = A tumor-targeting peptide GE11 (F-GE11) is conjugated to the last residue of the peptide.
GGGGSQPLGLVAKLSLITRLGGGGSQPLGLVAKTRSAWGGGGSQPLGLVAK

SEQ ID NO: 65 - protein, 40 aa, synthetic construct
  MOD_RES 1: note = A Secrecon peptide (Sec) is conjugated to residue 1.
  MOD_RES 40: note = A tumor-targeting peptide GE11 (F-GE11) is conjugated to the last residue of the peptide.
QPLGLVAKLSLITRLQPLGLVAKTRSAWGRSGQPLGLVAK

SEQ ID NO: 66 - protein, 40 aa, synthetic construct
  MOD_RES 1: note = A Secrecon peptide (Sec) is conjugated to residue 1.
  MOD_RES 40: note = A tumor-targeting peptide GE11 (F-GE11) is conjugated to the last residue of the peptide.
QPLGLVAKLSLITRLQPLGLVAKTRSAWQPLGLVAKGRSG

SEQ ID NO: 67 - protein, 44 aa, synthetic construct
  MOD_RES 1: note = A Secrecon peptide (Sec) is conjugated to residue 1.
  MOD_RES 44: note = A tumor-targeting peptide GE11 (F-GE11) is conjugated to the last residue of the peptide.
QPLGLVAKLSLITRLQPLGLVAKTRSAWGRSGQPLGLVAKGRSG

SEQ ID NO: 68 - protein, 40 aa, synthetic construct; identical to SEQ ID NO: 65
  MOD_RES 1: note = A Secrecon peptide (Sec) is conjugated to residue 1.
  MOD_RES 40: note = A tumor-targeting peptide GE11 (R-GE11) is conjugated to the last residue of the peptide.

SEQ ID NO: 69 - protein, 40 aa, synthetic construct; identical to SEQ ID NO: 66
  MOD_RES 1: note = A Secrecon peptide (Sec) is conjugated to residue 1.
  MOD_RES 40: note = A tumor-targeting peptide GE11 (R-GE11) is conjugated to the last residue of the peptide.

SEQ ID NO: 70 - protein, 48 aa, synthetic construct
  MOD_RES 1: note = A Secrecon peptide (Sec) is conjugated to residue 1.
QPLGLVAKLSLITRLQPLGLVAKTRSAWQPLGLVAKWQPDTAHHWATL

SEQ ID NO: 71 - protein, 48 aa, synthetic construct
  MOD_RES 1: note = A Secrecon peptide (Sec) is conjugated to residue 1.
QPLGLVAKLSLITRLQPLGLVAKTRSAWQPLGLVAKKYLAYPDSVHIW

SEQ ID NO: 72 - protein, 48 aa, synthetic construct; identical to SEQ ID NO: 71
  MOD_RES 1: note = A Secrecon peptide (Sec) is conjugated to residue 1.

SEQ ID NO: 73 - protein, 43 aa, synthetic construct
  MOD_RES 1: note = A Secrecon peptide (Sec) is conjugated to residue 1.
QPLGLVAKLSLITRLQPLGLVAKTRSAWQPLGLVAKSNTRVAP

SEQ ID NO: 74 - protein, 47 aa, synthetic construct
  MOD_RES 1: note = A Secrecon peptide (Sec) is conjugated to residue 1.
GRSGQPLGLVAKLSLITRLQPLGLVAKTRSAWQPLGLVAKSNTRVAP

SEQ ID NO: 75 - protein, 51 aa, synthetic construct
  MOD_RES 1: note = A Secrecon peptide (Sec) is conjugated to residue 1.
GRSGQPLGLVAKLSLITRLQPLGLVAKTRSAWGRSGQPLGLVAKSNTRVAP

SEQ ID NO: 76 - protein, 51 aa, synthetic construct
  MOD_RES 1: note = A Secrecon peptide (Sec) is conjugated to residue 1.
QPLGLVAKGRSGLSLITRLQPLGLVAKTRSAWQPLGLVAKGRSGSNTRVAP

(19) United States
(12) Patent Application Publication
(10) Pub. No.: US 2026/0308217 A1
(43) Pub. Date: Oct. 8, 2026

(54) A CYTOKINE-LIKE PROPEPTIDE GENE THERAPY PLATFORM

(71) Applicant: Purdue Research Foundation, West Lafayette, IN (US)

(72) Inventor: Marxa L. Figueiredo, West Lafayette, IN (US)

(21) Appl. No.: 19/484,806

(22) PCT Filed: May 15, 2024
(86) PCT No.: PCT/US2024/029425

§ 371 Date: Nov. 14, 2025

Related U.S. Application Data
(60) Provisional application No. 63/502,170, filed on May 15, 2023.

(51) Int. Cl. C07K 14/52; A61K 38/19; A61K 48/00; A61P 19/02; A61P 35/04

(52) U.S. Cl. CPC C07K 14/52; A61K 38/19; A61K 48/0033; A61K 48/005; A61P 19/02; A61P 35/04; C07K 2319/60

(57) ABSTRACT

One of the greatest challenges of current prostate cancer bone metastases treatments is the simultaneous tumor growth reduction or elimination while restoring bone homeostasis. This disclosure describes the development of a cytokine-like gene therapy class of molecules called propeptides that can be used to achieve these therapeutic goals. Propep is a multifunctional biologic with a pro-osteogenic module and anti-tumorigenic or anti-inflammatory modules. It can be secreted from cells because it contains a peptide secretion signal. It can be targeted also and anchored at the site of metastases, because it contains a tumor-homing peptide sequence. Propep is highly modular and can be utilized for several therapeutic applications, including: anti-inflammatory, anti-tumorigenic, and pro-osteogenic applications.

### CROSS-REFERENCE TO RELATED APPLICATION(S)

The present disclosure claims priority to U.S. Provisional Patent Application Ser. No. 63/502,170, filed May 15, 2023, which is hereby incorporated by reference in its entirety.

### INCORPORATION OF SEQUENCE LISTING

A computer readable form of the Sequence Listing XML containing the file named “3526450.0008 Sequence Listing.xml”, which is 114,628 bytes in size (as measured in MICROSOFT WINDOWS® EXPLORER) and was created on May 7, 2024, are provided herein and are herein incorporated by reference. This Sequence Listing consists of SEQ ID Nos: 1-76.

### FIELD OF DISCLOSURE

The present disclosure is directed to a cytokine-like gene therapy class of molecules called propeptides that can be utilized for several therapeutic applications, including: anti-inflammatory, anti-tumorigenic, and pro-osteogenic applications. Propep is a multifunctional biologic with a pro-osteogenic module and either anti-tumorigenic or anti-inflammatory modules. It can be secreted from cells because it contains a peptide secretion signal. It can be targeted also and anchored at the site of metastases because it contains a tumor-homing peptide sequence.

### BACKGROUND OF DISCLOSURE

Prostate cancer (PCa) arises from uncontrollable cell growth and division that originates in the prostate gland and could spread into surrounding tissue through lymph nodes. Second to skin cancer, prostate cancer is the most common cancer in men. The National Cancer Institute estimates 174,650 new cases, and 31,620 deaths from prostate cancer for 2019. 1 in 9 men will be diagnosed with prostate cancer in their lifetime, and 6 in 10 cases are diagnosed in men aged 65 or older. 1 in 41 men will succumb to prostate cancer. Fortunately, there has been a steady decline in prostate cancer new cases and death numbers over the past two decades, with the overall five-year survival rate now being at 97.8%. This five-year survival rate, however, is highly dependent on the stage at which the disease is diagnosed, with only a 30.5% survival rate once the disease is staged as distant from the primary site (i.e., metastatic). Prostate cancer can metastasize to any distant organ, but approximately 90% of patients that succumb to PCa have had bone metastases.

One of the greatest challenges of current prostate cancer bone metastases treatments is the simultaneous tumor growth reduction or elimination while restoring bone homeostasis. In the present disclosure, propeptide (cytokine-mimic) gene therapy in in vitro models is examined.

The overall design is that plasmids (gene therapy) expressing a propeptide therapeutic (Propep) are delivered to muscle cells, followed by production and secretion of the propeptide, which would contain a targeting moiety to enable it to accumulate at the disease site (bone metastases) and a pro-osteogenic moiety to promote rebalancing of bone remodeling. Critical to the success of this cytokine-mimic is the inclusion of MMP cleavage sites to process and separate Propep into its individual modules that bind different receptors. This is a highly modular and promising class of therapeutics that may be able to mimic cytokine activity in a multifunctional way, enabling treatment of complex microenvironments such as tumor metastases in bone.

### SUMMARY OF DISCLOSURE

This disclosure is directed to a propeptide comprising a secretion peptide, a pro-osteogenic peptide, an anti-tumorigenic peptide, and a tumor targeting peptide, wherein the secretion peptide comprises Secrecon comprising amino acid sequence MWWRLWWLLLLLLLLWPMVWA (SEQ ID NO:1); wherein the tumor targeting peptide comprises pepG comprising the GRP78 targeting peptide SNTRVAP (SEQ ID NO:2); wherein each peptide is separated by a matrix metalloprotease (MMP) cleavage site comprising amino acid sequence GPLGMLSQ (SEQ ID NO:3) or a prostate specific antigen (PSA) cleavage site comprising amino acid sequence RSSYYSL (SEQ ID NO:4).

Another aspect of the disclosure is a propeptide comprising a secretion peptide, a pro-osteogenic peptide, an anti-inflammatory peptide, and a joint targeting peptide, wherein the secretion peptide comprises Secrecon comprising amino acid sequence MWWRLWWLLLLLLLLWPMVWA (SEQ ID NO:1); wherein the joint targeting peptide comprises pepL comprising the IL-6Rα targeting peptide LSLITRL (SEQ ID NO:5); wherein each peptide is separated by a matrix metalloprotease (MMP) cleavage site comprising amino acid sequence GPLGMLSQ (SEQ ID NO:3).

The disclosure is further directed to DNA sequences encoding the disclosed propeptides and plasmids encoding these DNA sequences. The disclosure is also directed to gene therapies comprising these DNA sequences or plasmids.

A further aspect of the disclosure is a method of treating prostate cancer bone metastases in a subject in need thereof, the method comprising administering the gene therapy, disclosed herein encoding a propeptide comprising a secretion peptide, a pro-osteogenic peptide, an anti-tumorigenic peptide, and a tumor targeting peptide, to a muscle near the metastases, wherein the propeptide is produced and secreted from muscle cells in the muscle; wherein the propeptide is targeted to the metastases by the tumor targeting peptide; wherein the propeptide is cleaved into the secretion peptide, the pro-osteogenic peptide, the anti-tumorigenic peptide, and the tumor targeting peptide by MMPs found at the metastases; wherein the pro-osteogenic peptide restores bone homeostasis; and wherein the anti-tumorigenic peptide suppresses growth of the metastases.

Another aspect of the disclosure is a method of treating rheumatoid arthritis in a subject in need thereof, the method comprising administering the gene therapy, disclosed herein encoding a propeptide comprising a secretion peptide, a pro-osteogenic peptide, an anti-inflammatory peptide, and a joint targeting peptide, to a muscle near an arthritic joint in the subject, wherein the propeptide is produced and secreted from muscle cells in the muscle; wherein the propeptide is targeted to the arthritic joint by the joint targeting peptide; wherein the propeptide is cleaved into the secretion peptide, the pro-osteogenic peptide, the anti-inflammatory peptide, and the joint targeting peptide by MMPs found at the joint; wherein the pro-osteogenic peptide restores bone homeostasis; and wherein the anti-inflammatory peptide suppresses inflammation in the joint.

Unless otherwise defined, all technical and scientific terms used herein have the same meaning as commonly understood by one of ordinary skill in the art to which this disclosure pertains. Although methods and materials similar or equivalent to those described herein can be used to practice the invention, suitable methods and materials are described below. All publications, patent applications, patents, and other references mentioned herein are incorporated by reference in their entirety unless stated otherwise. In case of conflict, the present specification, including definitions, will control. In addition, the materials, methods, and examples are illustrative only and not intended to be limiting.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1A depicts a schematic representation of Propep modules. Propep and targeted IL-27 share modules with overlapping functions. pepL is a dual functional module, serving as an anti-tumorigenic module in Propep, and as a targeting module in targeted IL-27. ↓ indicates MMP cleavage sites.

FIG. 1B depicts RT-qPCR gene expression analysis of Ori-Propep.

FIG. 1C depicts a Western Blot of Flag-tagged Ori-pep, Flag-BAP positive control.

FIG. 1D depicts a fluorescent image of SDS-PAGE gel of C2C12 Click-Ori-Propep lysate. Ori-Propep is ~9 kDa. Transfection efficiency troubleshooting with Lipofectamine 3000 (L3000) or LTX, low (L) and high (H) ranges recommended by manufacturer. Fluorescent reporter is TAMRA (ex/em=557/583) Lane 1: Chameleon Duo Ladder; 2: No protein; 3: L3000-L+TAMRA; 4: L3000-L−TAMRA; 5: L3000-H+TAMRA; 6: L3000-H−TAMRA; 7: LTX3000-L+TAMRA; 8: LTX-L−TAMRA; 9: LTX-H+TAMRA; 10: Blank

FIG. 2 depicts a schematic representation of the selection and in silico optimization of targeting and functional modules of Propep.

FIGS. 3A-3E depict in silico modeling of Propeptide. Table 1 and 2 include a full list of Propep design models.

FIG. 3A depicts NoLinker EFWD, representing the secondary structure of the original Propep design.

FIG. 3B depicts NoLinker EREV, representing the secondary structure of the original Propep design.

FIG. 3C depicts 2X3X Linker EREV.

FIG. 3D depicts NoLinker-M2F, representing the 2nd generation Propep models. Propep D was selected for synthesis but failed QA/QC (Biomatik).

FIGS. 3E-3H depicts secondary structure prediction of 3rd generation Propep design incorporating secretion and targeting modules as well as matrix metalloproteases cleavage sites with linkers to enhance solvent accessibility. Propep H was selected for gene therapy development.

FIGS. 4A-4E depict Propeptide expression analysis.

FIGS. 4A-4C depict flow cytometry analysis of C2C12 (~1×10⁶) cells expressing Propep. Fusion with GFP allowed for the quantification of C2C12 cells expressing Propep-GFP.

FIG. 4D depicts RT-qPCR analysis of Propep and ClickiT-Propep expression in C2C12 cells.

FIG. 4E depicts a Western blot analysis of Propep expression in E. coli. NC: cell lysate without induction. Lanes 1 and 2: cell lysate with induction for 16-hrs at 15° C. and 4-hrs at 37° C., respectively. Lanes 3 and 4: supernatant of cell lysates with induction for 16-hrs at 15° C. and 4-hrs at 37° C., respectively. Lanes 5 and 6: pellet of cell lysates with induction for 16-hrs at 15° C. and 4-hrs at 37° C., respectively.

FIG. 5 depicts validation of rhMMP-9 Propep processing using fluorescently quenched peptides. ES001 is a commercially available peptide containing Mca/Dnp FRET pairs (R&D) utilized as a positive control. 20 ng of rhMMP-9 were used in each assay. Both PF0 and PF1 were custom ordered (Biomatik) and contain Propep targeting modality (pepG) and pro-bone modality (pepB). Only PF1 contains the recognition site for MMP-9 processing.

FIGS. 6A-6B depict rhMMP-9 processing of fluorescently quenched peptide ES001 (R&D) in conditioned media.

FIG. 6A depicts assessment of ES001 peptide processing in C4-2B cancer cells conditioned media. PAR-2 compound (Santa Cruz Biotechnology, AC 264613) suspended in DMSO has been shown to increase indigenous MMP-9 secreted from cancer cells. DMSO is a vehicle control.

FIG. 6B depicts assessment of ES001 peptide processing in C4-2B cancer cells conditioned media in the presence and absence of rhMMP-9. Exogenous rhMMP-9 remains active in conditioned media. ES001 peptide contains a recognition site for MMP-9 processing.

FIGS. 7A-7B depicts rhMMP-9 processing of fluorescently quenched control peptide PF0 (Biomatik) in C4-2B Conditioned Media.

FIG. 7A depicts assessment of PF0 peptide processing in C4-2B cancer cells conditioned media. PAR-2 compound (Santa Cruz Biotechnology, AC 264613) suspended in DMSO has been shown to increase indigenous MMP-9 from cancer cells. DMSO is a vehicle control.

FIG. 7B depicts assessment of PF0 peptide processing in C4-2B cancer cells conditioned media in the presence and absence of rhMMP-9. Exogenous rhMMP-9 remains active in conditioned media. PF0 peptide does not contain a recognition site for MMP-9 processing.

FIGS. 8A-8B depict rhMMP-9 processing of fluorescently quenched peptide PF1 (Biomatik) in C4-2B Conditioned Media.

FIG. 8A depicts assessment of PF1 peptide processing in C4-2B cancer cells conditioned media. PAR-2 compound (Santa Cruz Biotechnology, AC 264613) suspended in DMSO has been shown to increase indigenous MMP-9 from cancer cells. DMSO is a vehicle control.

FIG. 8B depicts assessment of PF1 peptide processing in C4-2B cancer cells conditioned media in the presence and absence of rhMMP-9. Exogenous rhMMP-9 remains active in conditioned media. PF1 peptide contains a recognition site for MMP-9 processing.

FIGS. 9A-9F depict FACS analysis of GRP78 and IL-6Ra expression on surface of C4-2B cancer cells. Thapsigargin (Tg) stimulates ER stress resulting GRP78 translocation to the cell surface, and it was used in the experiment described herein to better model the disease state of metastatic prostate cancer.

FIGS. 9A-9C depict GRP78 expression on C4-2B cells surface.

FIG. 9B is in the absence of Thapsigargin (Tg).

FIG. 9C is in the presence of Thapsigargin (Tg).

FIGS. 9D-9F depict IL-6Ra expression on C4-2B cells surface.

FIG. 9E is in the absence of Thapsigargin (Tg).

FIG. 9F is in the presence of Thapsigargin (Tg).

FIG. 10 depicts GRP78 targeting in C4-2B cells using FITC-labeled Propep (pepG). FACS analysis was used to evaluate targeting of FITC-labeled Propep in C4-2B cells following 16-hrs of stimulation with Thapsigargin (Tg). FACS analysis with a-Grp78 antibody indicated that ~20-30% of C4-2B cells expressed GRP78 on their cells surface, refer to FIGS. 9A-9F.

FIG. 11 depicts STAT-1 and STAT-3 activation in C4-2B cells following treatment with Propep conditioned media. Treatment of C4-2B cells with Propep conditioned media induced significant STAT-1 activation but did not induce any significant STAT-3 activation relative to controls.

FIGS. 12A-12B depict how Propep may have pro-osteogenic activity through increasing OB differentiation and reducing OC differentiation.

FIG. 12A depicts differentiating osteoclasts, day 6, using conditioned media from C2C12 cells to mimic gene delivery in vivo from skeletal muscle transfection. *p<0.05, **p<0.005, two-tailed t-test.

FIG. 12B depicts differentiating osteoblasts, day 21, by alizarin red staining. *p<0.05, one-tailed t-test.

FIG. 13 depicts how ProPep structures can contain dual therapeutic domains composed of an anti-inflammatory and an osteogenesis-promoting (or anti-resorptive) module.

### DETAILED DESCRIPTION OF DISCLOSURE

The present disclosure is directed to the development of a cytokine-like gene therapy class of molecules called propeptides. Propep is a multifunctional biologic with a pro-osteogenic module and one or more of anti-tumorigenic or anti-inflammatory modules. It can be secreted from cells because it contains a peptide secretion signal. It can be targeted also and anchored at the site of metastases because it contains a tumor-homing peptide sequence. Propep is highly modular and can be utilized for several therapeutic applications, including: anti-inflammatory, anti-tumorigenic, and pro-osteogenic.

In more detail, this disclosure covers the concept and data relating to the development of a therapeutic platform called Propeptides or ProPep in short. These can have a variety of modules, including IL6Ra targeting peptide, GRP78 targeting peptide, TRSAW or other proosteogenic peptides (BMP7 mimic, OPG, OSM) and anti-inflammatory peptides (CSF2R, IL1A). These modules are built into the ProPep with cleavable sites in between them to enable their release at the site of tumor metastases or inflammation. Critical to the success of this cytokine-mimic is the inclusion of MMP (matrix metalloproteases) or PSA (prostatic specific antigen) cleavage sites to process and separate Propep into its individual modules that bind different receptors.

The ProPep can be produced via gene delivery of muscle or gene delivery to adipose stromal cells, followed by secretion by these cells.

One of the greatest challenges of current prostate cancer bone metastases treatments is the simultaneous tumor growth reduction or elimination while restoring bone homeostasis. Cytokine-based therapeutics called propeptides can be used to achieve these therapeutic goals. The overall design is that plasmids (gene therapy) expressing a propeptide therapeutic (Propep) would be delivered to muscle cells, followed by production and secretion of the propeptide, which would contain a targeting moiety to enable it to accumulate at the disease site (bone metastases) and a pro-osteogenic moiety to promote rebalancing of bone remodeling. Critical to the success of this cytokine-mimic is the inclusion of MMP (matrix metalloproteases) or PSA (prostatic specific antigen) cleavage sites to process and separate Propep into its individual modules that bind different receptors.

This therapy can be directed to prostate cancer, bone therapy, and arthritis.

This disclosure is directed to a propeptide comprising a secretion peptide, a pro-osteogenic peptide, an anti-tumorigenic peptide, and a tumor targeting peptide, wherein the secretion peptide comprises Secrecon comprising amino acid sequence MWWRLWWLLLLLLLLWPMVWA (SEQ ID NO:1); wherein the tumor targeting peptide comprises pepG comprising the GRP78 targeting peptide SNTRVAP (SEQ ID NO:2); wherein each peptide is separated by a matrix metalloprotease (MMP) cleavage site comprising amino acid sequence GPLGMLSQ (SEQ ID NO:3) or a prostate specific antigen (PSA) cleavage site comprising amino acid sequence RSSYYSL (SEQ ID NO:4). In an alternative to MMP sites, PSA sites (prostate specific antigen sites) can be used, which are upregulated at prostate tumor bone metastases. The anti-tumorigenic peptide can comprise pepL comprising amino acid sequence LSLITRL (SEQ ID NO:5). The pro-osteogenic module can comprise pepB comprising osteostatin peptide TRSAW (SEQ ID NO:6). The propeptide can comprise or consist of the amino acid sequence of SecGRSG-GRP78 or ScGRSG1.3-GPR78.

Another aspect of the disclosure is a propeptide comprising a secretion peptide, a pro-osteogenic peptide, an anti-inflammatory peptide, and a joint targeting peptide, wherein the secretion peptide comprises Secrecon comprising amino acid sequence MWWRLWWLLLLLLLLWPMVWA (SEQ ID NO:1); wherein the joint targeting peptide comprises pepL comprising the IL-6Rα targeting peptide LSLITRL (SEQ ID NO:5); wherein each peptide is separated by a matrix metalloprotease (MMP) cleavage site comprising amino acid sequence GPLGMLSQ (SEQ ID NO:3). The pro-osteogenic module can comprise pepB comprising osteostatin peptide TRSAW (SEQ ID NO:6).

The propeptide can further comprise a reporter. The reporter can comprise ClickiT or GFP. The propeptide can further comprise amino acid sequence PAS. The sequence PAS (pro ala ser) sequence can enhance its half-life in vivo from a gene delivery or protein delivery perspective.

The propeptide can further comprise linkers or spacer peptides that enhance the solvent accessibility of the MMP or PSA cleavage sites. The MMP cleavage site can be a MMP-9 cleavage site.

The disclosure is further directed to DNA sequences encoding the disclosed propeptides and plasmids encoding these DNA sequences. The disclosure is also directed to gene therapies comprising these DNA sequences or plasmids.

A further aspect of the disclosure is a method of treating prostate cancer bone metastases in a subject in need thereof, the method comprising administering the gene therapy, disclosed herein encoding a propeptide comprising a secretion peptide, a pro-osteogenic peptide, an anti-tumorigenic peptide, and a tumor targeting peptide, to a muscle near the metastases, wherein the propeptide is produced and secreted from muscle cells in the muscle; wherein the propeptide is targeted to the metastases by the tumor targeting peptide; wherein the propeptide is cleaved into the secretion peptide, the pro-osteogenic peptide, the anti-tumorigenic peptide, and the tumor targeting peptide by MMPs found at the metastases; wherein the pro-osteogenic peptide restores bone homeostasis; and wherein the anti-tumorigenic peptide suppresses growth of the metastases. The prostate cancer can be androgen-independent prostate cancer or metastatic castration resistant prostate cancer.

Another aspect of the disclosure is a method of treating rheumatoid arthritis in a subject in need thereof, the method comprising administering the gene therapy, disclosed herein encoding a propeptide comprising a secretion peptide, a pro-osteogenic peptide, an anti-inflammatory peptide, and a joint targeting peptide, to a muscle near an arthritic joint in the subject, wherein the propeptide is produced and secreted from muscle cells in the muscle; wherein the propeptide is targeted to the arthritic joint by the joint targeting peptide; wherein the propeptide is cleaved into the secretion peptide, the pro-osteogenic peptide, the anti-inflammatory peptide, and the joint targeting peptide by MMPs found at the joint; wherein the pro-osteogenic peptide restores bone homeostasis; and wherein the anti-inflammatory peptide suppresses inflammation in the joint.

Administering the gene therapy can comprise administration via any gene delivery platform known in the art, including 1) nonviral gene delivery to muscle, or 2) viral gene delivery systems to muscle, or 3) mesenchymal stromal cell delivery in vivo. Administering the gene therapy can comprise administration via adeno-associated virus/phage or plasmid. The adeno-associated virus/phage can be AAV or Ad5.

As used in this application, including the appended claims, the singular forms “a,” “an,” and “the” include plural references unless the content clearly dictates otherwise, and are used interchangeably with “at least one” and “one or more.”

The disclosure will be further described in the following examples, which do not limit the scope of the disclosure described in the claims.

### EXAMPLES

It is to be understood that while the disclosure has been described in conjunction with the detailed description thereof, the preceding description is intended to illustrate and not limit the scope of the disclosure, which is defined by the scope of the appended claims. Other aspects, advantages, and modifications are within the scope of the following claims.

### Example 1: Materials and Methods

### In Silico Modeling of Propep

The I-TASSER server¹²⁶⁻²⁸ was used to predict the secondary structure of Propep models and aid in the selection of the most favorable design. Selection criteria included MMP recognition sites, spaces/linkers sequences and length, secretion modules, and targeting modules.

### Vectors

PCR cloning was utilized to amplify Propeptide gene from a custom plasmid SecGRSG1.3GRP78 in pUC57 (Biomatik). pMONO-LGFP (InvivoGen, pmonon-gfp) was used as the vector backbone and was linearized using restriction enzymes BmgBI to generate the backbone for Propep-GFP or BspHI/AvrII to generate the backbone for Propep and Propep-ClickiT. HiFi DNA assembly cloning kit (New England Biolabs, E5520) was used to generate Propeptide plasmids (Propep, Propep-ClickiT, and Propep-GFP). All plasmids were extracted using GeneJET plasmid midiprep kits (ThermoFisher; K0482). The primer sequences for the amplification and assembly are: Propep—F: 5′-CAAAGCAACCGGTCGCACGTCATGAGCC ACCATGTGGTGGAGG-′3 (SEQ ID NO:7). R: 5′-GTCATTGGGGAAACCTGCTCCTAGGGGCTCATGGA GCCACCCT-′3 (SEQ ID NO:8). Propep-ClickiT—F: 5′-CAAAGCAACCGGTCGCACGTCATGAGCCACC ATGGGTAACGAAGCGTCTTACCCGCTGAGTGGTGGAGGCTGTGG-′3 (SEQ ID NO:9). R: 5′-GTCATT GGGGAAACCTGCTCCTAGGGGCTCATGGAGCCACCCT-′3 (SEQ ID NO: 10). Propep-GFP—F: 5′-TAAT TCAAAGCAACCGGTCGTAGCCACCATGTGGTGGAGG-′3 (SEQ ID NO:11). R: 5′-AGTTCTTCTCCCTTG CTCATGCCGCTCGAGCCTG GAGCCACCCTGGTGTTG-′3 (SEQ ID NO: 12).

### C2C12 Cells Transfection and Conditioned Media Preparation

Lipofectamine 2000 (L2000) (Invitrogen, 11668030) was used to deliver Propeptide plasmids (pPropep, pClickIT-Propep, pPropep-GFP) C2C12 cells following manufacturer's protocol. However, a reverse transfection method was used to deliver plasmids to C2C12 cells. With this method, plasmids were complexed with Lipofectamine 2000, then added to each well in a 6-well plate. C2C12 cells were suspended in complete DMEM media (10% FBS, 1% anti/anti) and were then seeded into the 6-wells plates and mixed with the plasmid/L2000 complex and allowed to adhere and transfect for 24 hrs at 37° C. and 5% C02. Following 24-hr transfection, the media containing plasmids/L2000 was removed, and cells were washed with 1×DPBS (Corning, MT21031CV). The media was replaced with complete DMEM and cells were allowed to recover overnight. The next day, the cells were washed with 1×DPBS and media was replaced with complete RPMI (10% FBS, 1% anti/anti) and incubated with cells for 24-hrs to generate conditioned media (CM).

### Propep Expression Analysis

Fluorescence Activated Cell Sorting (FACS). C2C12 transfected with pPropep-GFP were lifted using 2 mM EDTA and suspended in cell staining buffer (BioLEGEND®, 420201). BD GOLGIPLUG™ (BD Biosciences, 555029) was used to inhibit secretion of Propep-GFP. Transfected C2C12 cells were treated with GOLGIPLUG™ for 4-6 hrs following manufacturer's protocol prior to preparation for FACS analysis. Each sample contained ~1×10⁶ C2C12 cells and the expression of pPropep-GFP was evaluated using Attune NTX flow cytometer (ThermoFisher).

### Gene Expression Analysis

RNA was isolated from C2C12 cell pellets using RNAeasy kit (Qiagen, Germantown, MD; 74104) according to manufacturer's protocols. 500 ng of RNA was reverse transcribed using AmfiRivert kit (Genedepot, Katy, TX; R5600-050), and 1 mL was used in SYBR green quantitative PCR (Fisher Scientific; NC9020838) reaction for detecting differences in gene expression relative to mouse b-actin. The primer pair used to detect Propep gene expression was custom designed containing the sequence F: 5′-GTC TGG ACA GCC TCT GGG A-′3 (SEQ ID NO: 13). R: 5′-TGG TGG CTA AGA GCA ACA CC-′3 (SEQ ID NO:14).

### Western Blot

E. coli BL21(DE3) competent cells were transformed with the recombinant Propep plasmid. A single colony was inoculated into LB medium containing kanamycin; cultures were incubated in 37° C. at 200 rpm. Once cell density reached to OD=0.6-0.8 at 600 nm, 0.5 mM IPTG was added for induction. SDS-PAGE and Western blot were used to monitor Propep expression.

Propep Processing with Matrix Metalloproteases

Recombinant human MMP-9 (rhMMP-9) (R&D systems, 911-MP) was activated by adding p-aminophenylmercuric acetate (APMA) to a final concentration of 1 mM following manufacturer's protocols and incubated at 37° C. for 24-hrs. Activated rhMMP-9 was diluted to 0.4 ng/mL in assay buffer (TCNB) (50 mM Tris, 10 mM CaCl₂), 150 mM NaCl, 0.05% Brij-35 (w/v), pH 7.5). In a black 96-well plate, 50 mL of activated rhMMP-9 containing 0.40 ng/mL was loaded into each well, and the reaction was initiated by adding 20 mM substrate: ES001 (Mca-PLGL-Dnp-Ar-NH2 (SEQ ID NO:15) (R&D Systems, ES001), PF0: Mca-TRSAWGRSGSNTRVAP-Lys(Dnp) (SEQ ID NO:16) (Biomatik, custom peptides), PF1: Mca-TRSAWGRSGQPLGLVAKSNTRVAP-Lys(Dnp) (SEQ ID NO:17) (Biomatik, custom peptides). Excitation and emission were measured at wavelengths of 320 nm and 405 nm in kinetic mode for 10, 12, or 120 minutes using a Synergy H1 Hybrid plate reader (BioTek).

Propep Targeting Analysis with FACS

C4-2B cells were growth in complete RPMI media (10% FBS, 1% anti/anti) at 37° C. and 5% CO₂. Once cells reached 80-90% confluency, they were treated with thapsigargin (Tg) (Tocris Biotechne; 1138) at 300 nM for 16-hrs to induce ER stress and the translocation of cytoplasmic Grp78 to the surface of C4-2B cells. 129 C4-2B were lifted using 2 mM EDTA and suspended in cell staining buffer (BIOLEGEND®) containing 0.1% sodium azide. Grp78-targeting peptide (pepG: FITC-Ahx-SNTRVAP (SEQ ID NO:18)) was custom ordered from Biomatik. PepG was reconstituted in sterile water at a stock concentration of 1 mg/mL (0.802 mM). Concentrations between 0-100 mM were used to evaluate targeting efficacy of pepG in samples containing ~1×10⁶ C4-2B cells. Cells were incubated with pepG on ice for 30 mins, washed 3× using cells staining buffer containing 0.1% sodium azide. Fluorescence was measured using Attune NTX flow cytometer. Nonlinear curve fitting (dose response) was processed using Origin 2018 software.

### Functional Assays

In a 24-well plate, 1×10⁵ C4-2B cells were seeded into each well and allowed to adhere overnight. Lipofectamine 2000 (ThermoFisher; L300015) was used to deliver 1 μg of plasmids responsive to active (phosphorylated) STAT-1 (pGAS/ISRE Luc) (Signosis; LR-2016) or STAT-3 (pSTAT-3-Luc) (Signosis; LR-2004) following the manufacturer's protocols. 1% b-galactosidase (pCMV-bgal) was used as a transfection control. Transfection media was aspirated, cells were washed with 1×DPBS, then cells were allowed to recover overnight in complete RPMI media (10% FBS, 1% anti/anti) 37° C. and 5% C02. Transfected C4-2B cells were serum starved for 5-6 hrs by replacing complete RPMI media with OptiMEM media (ThermoFisher, 11058021). Following serum starvation, C4-2B cells were treated with 1:1 complete RPMI and conditioned media containing Propep (secreted) for 35-hrs. Cells were then trypsinized then centrifuged at 1500 rpm for 5 mins to form pellets. To measure STAT-1/-3 activation, cells were lysed with 40 uL 1× passive lysis buffer, lysates kept on ice, then 50 uL luciferin substrate were added, and luminescence measured using GloMax plate reader (Promega) with a 10 second integration time.

### Statistical Analysis

Assays were performed in triplicate and values provided as mean±SEM. Comparisons were performed using unpaired t-tests or one-way analysis of variance analysis. A p-value <0.05 indicates a significant difference (*).

### Example 2: Propeptide Module Conceptualization

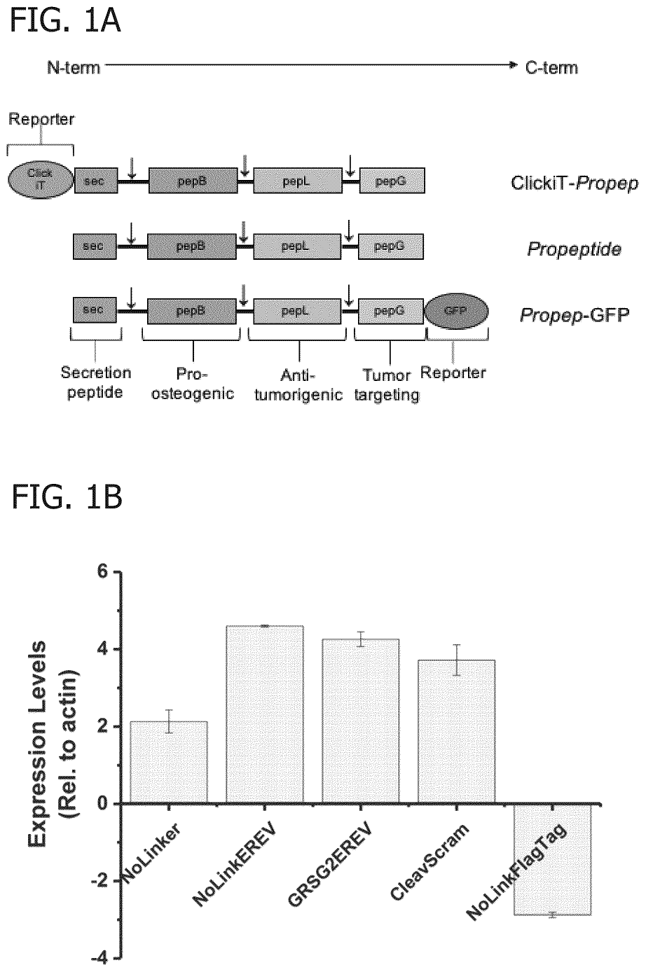

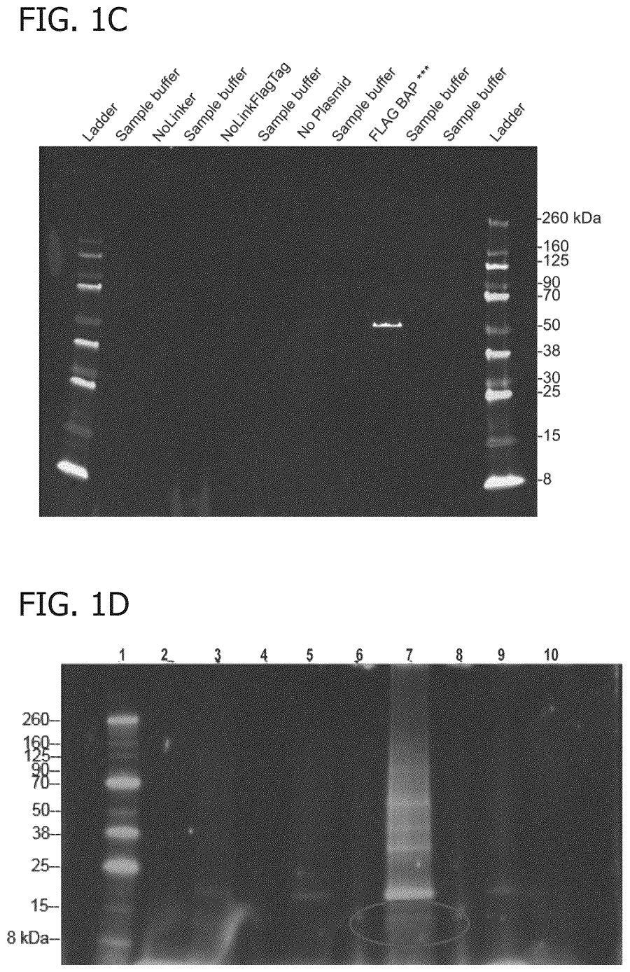

As illustrated in FIG. 1A, Propep is a multifunctional biologic with a pro-osteogenic module and an anti-tumorigenic module. It can be secreted from cells because it contains a peptide secretion signal. It can be targeted also and anchored at the site of metastases because it contains a tumor-homing peptide sequence. The original design of the ‘cytokine-based’ Propep (1st generation propep) therapeutic utilized signal peptide from tissue plasminogen activator (tPA) as the secretion motif, and the epidermal growth factor receptor (EGFR) targeting motif GE11102 or tumor targeting (Table 1):

TABLE 1
Optimization of Solvent Accessibility to MMP Cleavage
Sites via Linker Variations.

| | | | MMP-Site 1 | MMP-Site 2 | MMP-Site 3 | |
|---|---|---|---|---|---|---|
| | | | (GPLG↓MLSQ) | (GPLG↓MLSQ) | (GPLG↓MLSQ) | |
| | | | (SEQ ID | (SEQ ID | (SEQ ID | |
| No. | Propeptide | Sequence | NO: 3) | NO: 3) | NO: 3) | Comments |
| 1 | NoLinker | [tPA]GPLGMLSQLSLITRL | 5534↓4344 | 5434↓3334 | 2322↓2234 | Original |
| | | GPLGMLSQTRSAWGPLGMLS | | | | propeptides |
| | | Q[F-EGFR-A] | | | | |
| | | (SEQ ID NO: 22) | | | | |
| 2 | NoLinkEREV | [tPA]GPLGMLSQLSLITRL | 5534↓4344 | 5434↓3334 | 3323↓2134 | |
| | | GPLGMLSQTRSAWGPLGMLS | | | | |
| | | Q[R-EGFR-A] | | | | |
| | | (SEQ ID NO: 23) | | | | |
| 3 | GRSG2EREV | [tPA]GRSGGPLGMLSQLSL | 34444444 | 5534↓3334 | 2323↓2034 | |
| | | ITRLGPLGMLSQTRSAWGPL | | | | |
| | | GMLSQ[R-EGFR-A] | | | | |
| | | (SEQ ID NO: 24) | | | | |
| 4 | 3XLinkEREV | [tPA]GRSGGPLGMLSQLSL | 2222↓2243 | 2332↓3134 | 2223↓2034 | Δlinker |
| | | ITRLGRSGGPLGMLSQTRSA | | | | type and |
| | | WGRSGGPLGMLSQI[R-EGF | | | | repeats + |
| | | R-A] | | | | orientation of |
| | | (SEQ ID NO: 25) | | | | targeting |
| 5 | 2X3XLinkEREV | [tPA]GRSGGRSGGPLGMLS | 3132↓4454 | 4332↓4243 | 2223↓2034 | motif (C-term) |
| | | QLSLITRLGRSGGRSGGPLG | | | | Model #12 |
| | | MLSQTRSAWGRSGGRSGGPL | | | | was selected |
| | | GMLSQ | | | | for synthesis |
| | | [R-EGFR-A] | | | | |
| | | (SEQ ID NO: 26) | | | | |
| 6 | G3SLinkEREV | [tPA]GGGSGPLGMLSQLSL | 2222↓2143 | 2332↓3134 | 2223↓2034 | |
| | | ITRLGGGSGPLGMLSQTRSA | | | | |
| | | WGGGSGPLGMLSQ[R-EGFR | | | | |
| | | -A] | | | | |
| | | (SEQ ID NO: 27) | | | | |
| 7 | NoLinkMIR | [tPA]GPLGMLSQLSLITRL | 4333↓2133 | 3332↓2134 | 2223↓2034 | |
| | | GPLGMLSQGRSGTRSAWGRS | | | | |
| | | GGPLGMLSQ[R-EGFR-A] | | | | |
| | | (SEQ ID NO: 28) | | | | |
| 8 | NoLinkM1F | [tPA]GPLGMLSQLSLITRL | 4333↓2133 | 3332↓2134 | 2222↓2134 | |
| | | GPLGMLSQGRSGTRSAWGPS | | | | |
| | | GGPLGMLSQ[R-EGFR-A] | | | | |
| | | (SEQ ID NO: 29) | | | | |
| 9 | NoLinkM2R | [tPA]GPLGMLSQLSLITRL | 4333↓2133 | 3332↓2134 | 2222↓2134 | |
| | | GPLGMLSQGRSGTRSAWGRS | | | | |
| | | GGPLGMLSQGRSG[R-EGFR | | | | |
| | | -A] | | | | |
| | | (SEQ ID NO: 30) | | | | |
| 10 | NoLinkM2F | [tPA]GPLGMLSQLSLITRL | 4333↓2133 | 3332↓2134 | 2222↓2134 | |
| | | GPLGMLSQGRSGTRSAWGRS | | | | |
| | | GGPLGMLSQGRSG[F-EGFR | | | | |
| | | -A] | | | | |
| | | (SEQ ID NO: 31) | | | | |
| 11 | NoLinkM3R | [tPA]GPLGMLSQLSLITRL | 5634↓4344 | 5434↓3324 | 2332↓2134 | |
| | | GPLGMLSQTRSAWGRSGGPL | | | | |
| | | GMLSQGRSG[R-EGFR-A] | | | | |
| | | (SEQ ID NO: 32) | | | | |
| 12 | NoLinkM3F | [tPA]GPLGMLSQLSLITRL | 5634↓3344 | 5434↓3334 | 4434↓3145 | |
| | | GPLGMLSQTRSAWGRSGGPL | | | | |
| | | GMLSQGRSG[F-EGFR-A] | | | | |
| | | (SEQ ID NO: 33) | | | | |
| 13 | NoLinkM4R | [tPA]GPLGMLSQLSLITRL | 5634↓4344 | 5434↓3324 | 2233↓2034 | |
| | | GPLGMLSQTRSAWGRSGGPL | | | | |
| | | GMLSQ[R-EGFR-A] | | | | |
| | | (SEQ ID NO: 34) | | | | |
| 14 | NoLinkM4F | [tPA]GPLGMLSQLSLITRL | 4634↓4354 | 4434↓3334 | 2132↓2033 | |
| | | GPLGMLSQTRSAWGRSGGPL | | | | |
| | | GMLSQ[F-EGFR-A] | | | | |
| | | (SEQ ID NO: 35) | | | | |
| 15 | NoLinkM5R | [tPA]GPLGMLSQLSLITRL | 4333↓2133 | 2322↓2134 | 2222↓2134 | |
| | | GRSGGPLGMLSQGRSGTRSA | | | | |
| | | WGRSGGPLGMLSQGRSG[R- | | | | |
| | | EGFR-A] | | | | |
| | | (SEQ ID NO: 36) | | | | |
| 16 | NoLinkM5F | [tPA]GPLGMLSQLSLITRL | 4333↓2133 | 2322↓2134 | 2222↓2134 | |
| | | GRSGGPLGMLSQGRSGTRSA | | | | |
| | | WGRSGGPLGMLSQGRSG[F- | | | | |
| | | EGFR-A] | | | | |
| | | (SEQ ID NO: 37) | | | | |
| 17 | NoLinkM1, 1R | [tPA]GPLGMLSQLSLITRL | 4333↓2332 | 4232↓2044 | 2223↓2034 | |
| | | GRSGGPLGMLSQGRSGTRSA | | | | |
| | | WGRSGGPLGMLSQ[R-EGFR | | | | |
| | | -A] | | | | |
| | | (SEQ ID NO: 38) | | | | |

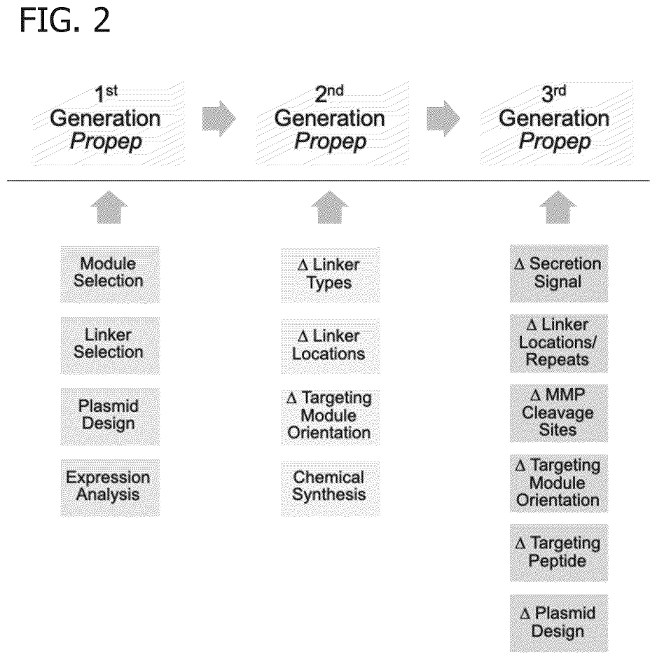

While the original therapeutic design exhibited high levels of gene expression in mouse myoblast muscle cells (C2C12), protein expression levels were undetectable using conventional analytical methods (western blot) (FIGS. 1B-1D). Furthermore, Biosystems Inc. was unsuccessful at producing a synthetic version of Ori-Propep. To overcome these setbacks, an extensive literature search was conducted for peptide sequences that could enhance expression levels and secretion from mouse muscle cells (FIG. 2 and Table 2).

TABLE 2
Propeptide Re-design and Solvent Accessibility Optimization.

| | | | MMP-Site 1 | MMP-Site 2 | MMP-Site 3 | |
|---|---|---|---|---|---|---|
| | | | (GPLG↓MLSQ) | (GPLG↓ MLSQ) | (GPLG↓MLSQ) | |
| | | | (SEQ ID | (SEQ ID | (SEQ ID | |
| No. | Propeptide | Sequence | NO: 3) | NO: 3) | NO: 3) | Comments |
| 1 | SecNoLink | [Sec]GPLGMLSQLSLITRL | 3333↓3243 | 3333↓3134 | 2322↓2234 | Targeting peptide |
| | | GPLGMLSQTRSAWGPLGMLS | | | | (tPA) |
| | | Q[F-GE11] | | | | replaced with |
| | | (SEQ ID NO: 42) | | | | Secrecon |
| 2 | Sec3XLink | [Sec]GRSGGPLGMLSQLSL | 2232↓2243 | 2332↓3134 | 2222↓2134 | (Sec) + Δ |
| | | ITRLGRSGGPLGMLSQTRSA | | | | linker type and |
| | | WGRSGGPLGMLSQ | | | | repeats |
| | | [F-GE11] | | | | |
| | | (SEQ ID NO: 43) | | | | |
| 3 | Sec2X3XLink | [Sec]GRSGGRSGGPLGMLS | 2222↓2243 | 2222↓2134 | 2222↓2134 | |
| | | QLSLITRLGRSGGRSGGRSG | | | | |
| | | GPLGMLSQTRSAWGRSGGRS | | | | |
| | | GG | | | | |
| | | RSGGPLGMLSQ[F-GE11] | | | | |
| | | (SEQ ID NO: 44) | | | | |
| 4 | SecG4Slink | [Sec]GGGGSGPLGMLSQLS | 2223↓2243 | 2222↓2034 | 2222↓2034 | |
| | | LITRLGGGGSGPLGMLSQTR | | | | |
| | | SAWGGGGSGPLGMLSQ[F-G | | | | |
| | | E11] | | | | |
| | | (SEQ ID NO: 45) | | | | |
| 5 | SecGGSLink | [Sec]GGSGGGSGGGPLGML | 2222↓2243 | 2222↓2134 | 2222↓2134 | |
| | | SQLSLITRLGGSGGGSGGGP | | | | |
| | | LGMLSQTRSAWGGSGGGSGG | | | | |
| | | GP | | | | |
| | | LGMLSQ[F-GE11] | | | | |
| | | (SEQ ID NO: 46) | | | | |
| 6 | SecNLM1Fwd | [Sec]GPLGMLSQLSLITRL | 3333↓3243 | 3332↓2134 | 2222↓2134 | |
| | | GPLGMLSQGRSGTRSAWGRS | | | | |
| | | GGPLGMLSQ[F-GE11] | | | | |
| | | (SEQ ID NO: 47) | | | | |
| 7 | SecNLM2Fwd | [Sec]GPLGMLSQLSLITRL | 3333↓3243 | 3332↓2134 | 2222↓3034 | |
| | | GPLGMLSQGRSGTRSAWGRS | | | | |
| | | GGPLGMLSQGRSG | | | | |
| | | [F-GE11] | | | | |
| | | (SEQ ID NO: 48) | | | | |
| 8 | SecNLM3Fwd | [Sec]GPLGMLSQLSLITRL | 3333↓3243 | 3333↓3134 | 2222↓3034 | |
| | | GPLGMLSQTRSAWGRSGGPL | | | | |
| | | GMLSQGRSG[F-GE11] | | | | |
| | | (SEQ ID NO: 49) | | | | |
| 9 | SecNLM4Fwd | [Sec]GPLGMLSQLSLITRL | 3333↓3243 | 3333↓3134 | 2232↓2134 | |
| | | GPLGMLSQTRSAWGRSGGPL | | | | |
| | | GMLSQ[F-GE11] | | | | |
| | | (SEQ ID NO: 50) | | | | |
| 10 | Sec3XLM1 | [Sec]GRSGGPLGMLSQLSL | 2232↓2243 | 3332↓2134 | 2222↓2134 | |
| | | ITRLGPLGMLSQGRSGTRSA | | | | |
| | | WGRSGGPLGMLSQGRSG[F- | | | | |
| | | GE11] | | | | |
| | | (SEQ ID NO: 51) | | | | |
| 11 | Sec3XLM2 | [Sec]GRSGGPLGMLSQLSL | 2222↓2143 | 2322↓2134 | 2222↓2034 | |
| | | ITRLGRSGGPLGMLSQGRSG | | | | |
| | | TRSAWGRSGGPLGMLSQGRS | | | | |
| | | G[F-GE11] | | | | |
| | | (SEQ ID NO: 52) | | | | |
| 12 | SecNL-LT | [Sec]PRSLTLSLITRLPRS | 344↓13 | 344↓14 | 344↓13 | ΔMMP cleavage |
| | | LTTRSAWPRSLT[F-GE11] | | | | site |
| | | (SEQ ID NO: 53) | | | | + linker |
| 13 | Sec3X-LT | [Sec]GRSGPRSLTLSLITR | 344↓13 | 344↓24 | 344↓13 | repeats |
| | | LGRSGPRSLTTRSAWGRSGP | | | | |
| | | RSLT[F-GE11] | | | | |
| | | (SEQ ID NO: 54) | | | | |
| 14 | Sec2X3X-LT | [Sec]GRSGGRSGPRSLTLS | 334↓12 | 344↓24 | 334↓13 | |
| | | LITRLGRSGGRSGPRSLTTR | | | | |
| | | SAWGRSGGRSGPRSLT[F-G | | | | |
| | | E11] | | | | |
| | | (SEQ ID NO: 55) | | | | |
| | | | MMP-Site 1 | MMP-Site 2 | MMP-Site 3 | |
| | | | (PLGILVA) | (PLG↓ LVA) | (PLG↓LVA) | |
| | | | (SEQ ID | (SEQ ID | (SEQ ID | |
| No. | Propeptide | Sequence | NO: 40) | NO: 40) | NO: 40) | Comments |
| 15 | SecNL-LVA | [Sec]GPLGLVAQLSLITRL | 3333↓2323 | 3332↓2124 | 22221113 | ΔMMP recognition |
| | | GPLGLVAQTRSAWGPLGLVA | | | | site + linker |
| | | Q[F-GE11] | | | | type and |
| | | (SEQ ID NO : 56) | | | | repeats |
| 16 | Sec3XLVA | [Sec]GRSGGPLGLVAQLSL | 2222↓1123 | 2321↓1124 | 222140103 | |
| | | ITRLGRSGGPLGLVAQTRSA | | | | |
| | | WGRSGGPLGLVAQ | | | | |
| | | [F-GE11] | | | | |
| | | (SEQID NO: 57) | | | | |
| 17 | Sec3XLVA-G | [Sec]GRSGPLGLVAQLSLI | 322241123 | 3322↓1024 | 2322↓1003 | |
| | | TRLGRSGPLGLVAQTRSAWG | | | | |
| | | RSGPLGLVAQ[F-GE11] | | | | |
| | | (SEQ ID NO: 58) | | | | |
| 18 | Sec2X3XLVA | [Sec]GRSGGRSGGPLGLVA | 2222↓1123 | 2221↓1124 | 2221↓0013 | |
| | | QLSLITRLGRSGGRSGGPLG | | | | |
| | | LVAQTRSAWGRSGGRSGGPL | | | | |
| | | GLV | | | | |
| | | AQ[F-GE11] | | | | |
| | | (SEQ ID NO: 59) | | | | |
| 19 | Sec2X3XLVA-G | [Sec]GRSGGRSGPLGLVAQ | 2322↓1123 | 2322↓1024 | 2322↓0003 | |
| | | LSLITRLGRSGGRSGPLGLV | | | | |
| | | AQTRSAWGRSGGRSGPLGLV | | | | |
| | | AQ | | | | |
| | | [F-GE11] | | | | |
| | | (SEQ ID NO: 60) | | | | |
| | | | MMP-Site 1 | MMP-Site 2 | MMP-Site 3 | |
| | | | (QPLG↓LVAK) | (QPLG↓ LVAK) | (QPLG↓LVAK) | |
| | | | (SEQ ID | (SEQ ID | (SEQ ID | |
| No. | Propeptide | Sequence | NO: 41) | NO: 41) | NO: 42) | Comments |
| 20 | SecNL-LVAK | [Sec]QPLGLVAKLSLITRL | 4323↓2123 | 4332↓2124 | 4222↓1104 | Δamino acid |
| | | QPLGLVAKTRSAWQPLGLVA | | | | residues |
| | | K[F-GE11] | | | | on either |
| | | (SEQ ID NO : 61) | | | | side of MMP |
| 21 | Sec3XLVAK | [Sec]GRSGQPLGLVAKLSL | 4222↓1123 | 4322↓1124 | 4222↓1014 | recognition |
| | | ITRLGRSGQPLGLVAKTRSA | | | | site |
| | | WGRSGQPLGLVAK | | | | |
| | | [F-GE11] | | | | |
| | | (SEQ ID NO: 62) | | | | |
| 22 | Sec2X3XLVAK | [Sec]GRSGGRSGQPLGLVA | 4222↓1123 | 4222↓1124 | 4222↓1014 | |
| | | KLSLITRLGRSGGRSGQPLG | | | | |
| | | LVAKTRSAWGRSGGRSGQPL | | | | |
| | | GLV | | | | |
| | | AK[F-GE11] | | | | |
| | | (SEQ ID NO: 63) | | | | |
| 23 | SecG4SLVAK | [Sec]GGGGSQPLGLVAKLS | 4222↓1123 | 4222↓1024 | 4222↓1004 | |
| | | LITRLGGGGSQPLGLVAKTR | | | | |
| | | SAWGGGGSQPLGLVAK | | | | |
| | | [F-GE11] | | | | |
| | | (SEQ ID NO: 64) | | | | |
| 24 | SecNL-LVAK1 | [Sec]QPLGLVAKLSLITRL | 4323↓2123 | 4322↓2124 | 4222↓1014 | Δ linker |
| | | QPLGLVAKTRSAWGRSGQPL | | | | locations ↓ |
| | | GLVAK[F-GE11] | | | | targeting |
| | | (SEQ ID NO: 65) | | | | peptide |
| 25 | SecNL-LVAK2 | [Sec]QPLGLVAKLSLITRL | 4323↓2123 | 4322↓2124 | 4322↓1124 | orientation |
| | | QPLGLVAKTRSAWQPLGLVA | | | | |
| | | KGRSG[F-GE11] | | | | |
| | | (SEQ ID NO: 66) | | | | |
| 26 | SecNL-LVAK3 | [Sec]QPLGLVAKLSLITRL | 432342123 | 4322↓2124 | 4222↓1024 | |
| | | QPLGLVAKTRSAWGRSGQPL | | | | |
| | | GLVAKGRSG[F-GE11] | | | | |
| | | (SEQ ID NO: 67) | | | | |
| 27 | SecNL-LVAKIR | [Sec]QPLGLVAKLSLITRL | 432342123 | 4322↓2124 | 4222↓1014 | |
| | | QPLGLVAKTRSAWGRSGQPL | | | | |
| | | GLVAK[R-GE11] | | | | |
| | | (SEQ ID NO: 68) | | | | |
| 28 | SecNL-LVAK2R | [Sec]QPLGLVAKLSLITRL | 4323↓2123 | 4322↓2124 | 43221124 | |
| | | QPLGLVAKTRSAWQPLGLVA | | | | |
| | | KGRSG[R-GE11] | | | | |
| | | (SEQ ID NO: 69) | | | | |
| 29 | SecNL-WQP | [Sec]QPLGLVAKLSLITRL | 4323↓2123 | 4332↓2124 | 4223↓2124 | Δ targeting |
| | | QPLGLVAKTRSAWQPLGLVA | | | | peptide |
| | | KWQPDTAHHWATL | | | | and linker |
| | | | | | | locations; |
| | | (SEQ ID NO: 70) | | | | top 2 |
| 30 | SecNL-KKYL | [Sec]QPLGLVAKLSLITRL | 4323↓2123 | 4322↓2124 | 4322↓2014 | candidates: |
| | | QPLGLVAKTRSAWQPLGLVA | | | | models 33 |
| | | KKYLAYPDSVHIW | | | | and 34 |
| | | (SEQ ID NO: 71) | | | | |
| 31 | SecNL-KYL | [Sec]QPLGLVAKLSLITRL | 4323↓2123 | 4322↓2124 | 4222↓2014 | |
| | | QPLGLVAKTRSAWQPLGLVA | | | | |
| | | KKYLAYPDSVHIW | | | | |
| | | (SEQ ID NO: 72) | | | | |
| 32 | SecNL-GRP78 | [Sec]QPLGLVAKLSLITRL | 4323↓2123 | 4332↓2124 | 4322↓1124 | |
| | | QPLGLVAKTRSAWQPLGLVA | | | | |
| | | KSNTRVAP | | | | |
| | | (SEQ ID NO: 73) | | | | |
| 33 | SecGRSG-GRP78 | [Sec]GRSGQPLGLVAKLSL | 4222↓1123 | 4332↓2124 | 4322↓1124 | |
| | | ITRLQPLGLVAKTRSAWQPL | | | | |
| | | GLVAKSNTRVAP | | | | |
| | | (SEQ ID NO: 74) | | | | |
| 34 | SecGRSG1.3- | [Sec]GRSGQPLGLVAKLSL | 4222↓1123 | 4322↓2124 | 4221↓1124 | |
| | GPR78 | ITRLQPLGLVAKTRSAWGRS | | | | |
| | | GQPLGLVAKSNTRVAP | | | | |
| | | (SEQ ID NO: 75) | | | | |
| 35 | SecGRSG1.3X- | [Sec]QPLGLVAKGRSGLSL | 4322↓2124 | 4322↓2124 | 4322↓1124 | |
| | GPR78 | ITRLQPLGLVAKTRSAWQPL | | | | |
| | | GLVAKGRSGSNTRVAP | | | | |
| | | (SEQ ID NO: 76) | | | | |

Several tumor-targeting peptides at the C-terminal end of Propep were also evaluated (FIG. 2 and Table 2). Finally, two peptide motifs were incorporated within Propep to stimulate pro-osteogenesis and anti-tumorigenesis signaling. Following these design criteria, a single cytokine-based Propep was produced: tumor-targeted secGRSG1.3-GRP78. Following completion of proof-of-concept experiments for the tumor targeted Propep the targeting and therapeutic efficacy of Propep will be validated in prostate cancer cell models.

### Example 3: Selection of the Tumor-Targeting Module of Propep

As the favorable response to standard androgen deprivation therapy is dramatically reduced with prostate cancer progression, researchers in the field have continued to search for molecular signatures that would allow for targeted delivery of therapeutic agents for combating prostate cancer metastases. Also, therapies are still needed that can help reduce the growth of androgen-independent tumors (metastatic castration resistant prostate cancer, mCRPC) in bone. Currently, prostate specific antigen (PSA) is widely used for diagnosis of prostate cancer, whereas prostate-specific membrane antigen (PSMA) is used for delivering treatment for CRPC. Unfortunately, the efficacy of both antigens is reduced by low accuracy due to them being organ-specific rather than tumor-specific biomarkers; therefore, there is some basal off-target activity towards the normal prostate.

Initially, there was interest in utilizing the epidermal growth factor receptor (EGFR) for targeting therapeutics to CRPC. This concept was based on the experimental evidence pointing to EGFR overexpression in 18% of prostate cancers categorized as advanced and with a high risk of recurrence and also in other studies reporting a 35% mean rate of EGFR expression, with rates spanning 1-86% for localized prostate cancer, and 16-100% in mPCa. Since this initial idea, it has been realized that this was a wide range of variability in EGFR expression in CRPC, suggesting that it may not be a reliable target. Although promising results have been shown in preclinical and clinical studies, where favorable anti-tumor effects in response to EGFR blockade with treatment with the monoclonal antibody (mAb) Cetuximab, increased survival was incomplete (only some patients), and doxorubicin was necessary as a concomitant therapy to achieve these survival increases. In addition to mAb, peptides also can be used as targeting moieties, since they display low immunogenicity and have the potential to penetrate solid tumors. The first-generation original design (Ori-Propep) utilized peptide GE11 (YHWYGYTPQNVI) (SEQ ID NO:19) as its tumor-targeting module (FIG. 2 and Table 1). The second generation Propep included linkers or spacer peptides to enhance the solvent accessibility of MMPs cleavage sites while maintaining the original peptide sequence of 1st generation Propep.

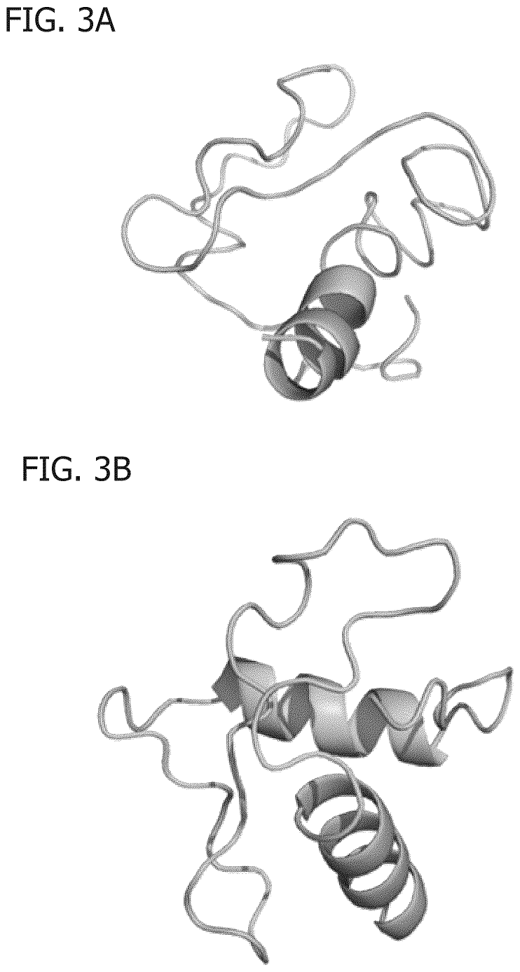

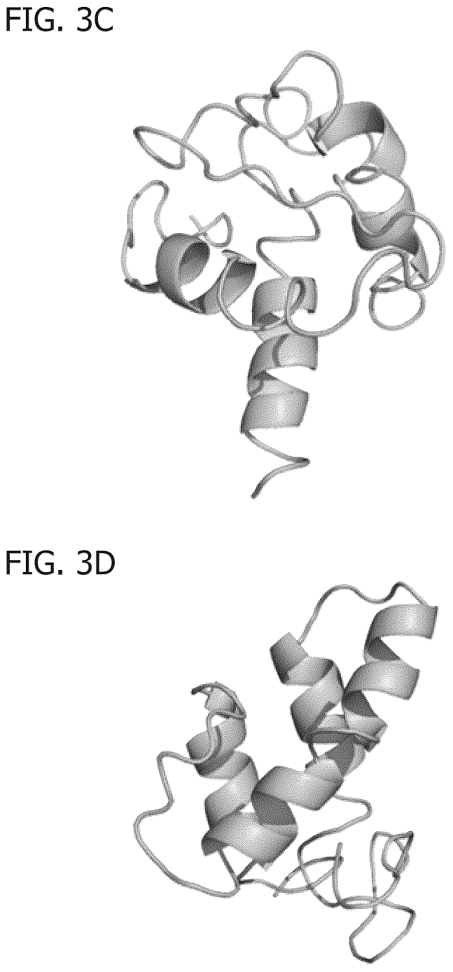

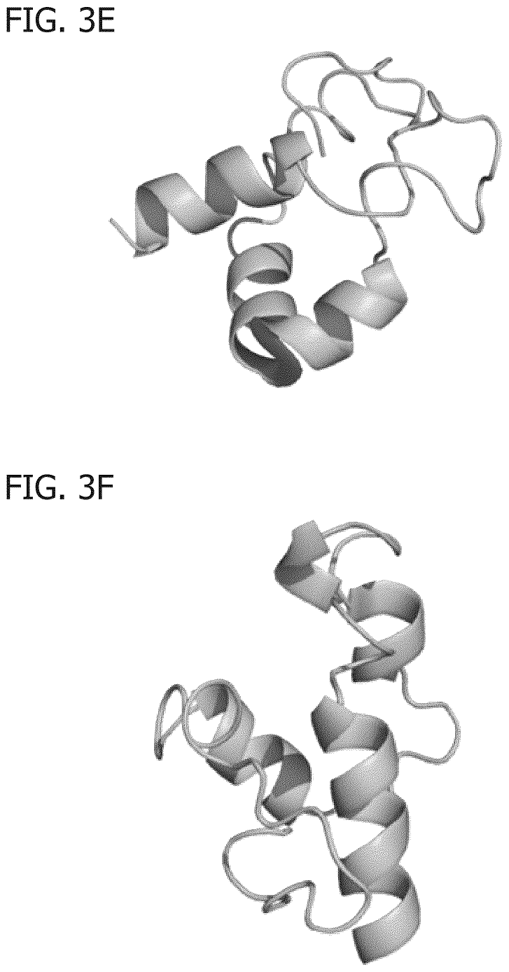

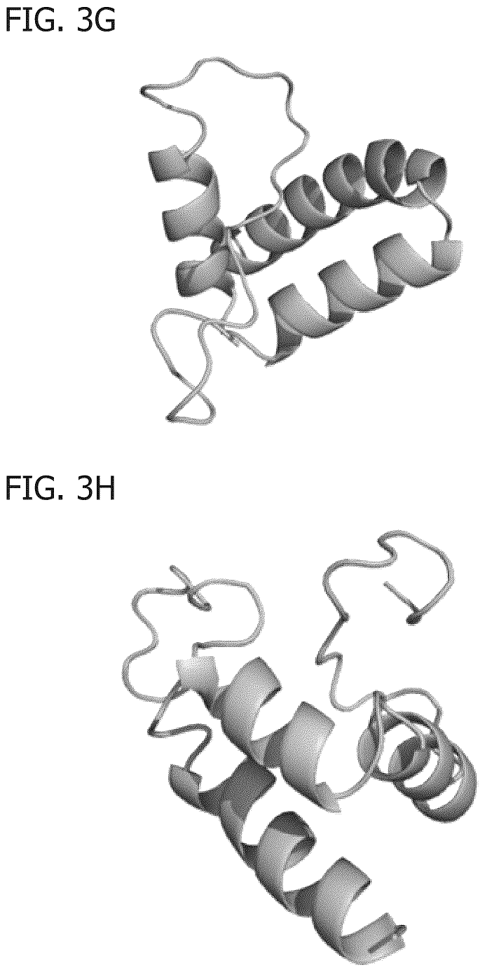

Chemical synthesis of both 1st and 2nd generation Propep were not successful, therefore the design of Propep was revisited in its entirety and the enhancement process began by selecting a new targeting module. The GE 11 peptide specifically binds to EGFR with a dissociation constant of ~22 nM; a significantly lower affinity for EGFR compared to its native ligand EGF (Kd=2 nM). This loss in affinity relative to EGF, combined with already established low and inconsistent expression levels of EGFR in mCRPC, ultimately suggested that GE11 could be a substandard module for mCRPC targeting. Therefore, a third-generation design was generated for an optimized Propep, replacing the targeting peptide with a peptide more recently described in mCRPC and bone that specifically binds to the glucose-regulated protein (GRP78) with high affinity and in a wide range of advanced mCRPC (dissociation constant not reported in literature), potentially being an improved targeting strategy (FIGS. 3A-3H and Table 2).

GRP78 is a stress response chaperone, and its upregulation has been shown to correlate with the development of CRPC, its recurrence, and low survival rates. Combinatorial peptide library screening and in vivo studies by the Arap and Pasqualini groups identified GRP78 as a prostate cancer antigen, validated its cell surface expression on CRPC cells, and reported that GRP78 ligands allow for specific targeting of solid tumors (breast and prostate cancers). 73% of untreated patients with localized prostate cancer displayed overexpression of Grp78, compared to overexpression in 67% of patients treated with ADT. In the highly bone mCRPC category, 100% of patients exhibited high GRP78 expression (28/28 CRPC patients overexpressed GRP78). Furthermore, GRP78 overexpression is associated with high risk of recurrence and low survival rates. Patients with low Grp78 expression had 14.5 recurrence-free years compared with 8.7 years in patients with high levels of Grp78 expression. Therefore, this work explored GRP78 targeting peptide (SNTRVAP) (SEQ ID NO:2) as the targeting module to deliver a muscle-secreted cytokine-like therapeutic agent (Propep) in prostate cancer cell models.

### Example 4: The Secretion Module of Propep

Secretion signal peptides (SP) are often incorporated into the design of recombinant proteins to overcome their refractory expression in mammalian systems. In the design of the therapeutic agents, the importance of signal peptides is two-fold. As mentioned above, secretion signal peptides enhance protein expression levels, which allows the use of skeletal muscle cells as production factories for achieving higher expression levels of secreted cytokine-based therapeutics. More importantly, this production and secretion scheme would allow for preclinical application of these therapeutic agents, where plasmid DNA coding of a specific therapeutic agent is delivered to muscles near the site of metastases, expressed in the muscles, secreted, then homed to PCa metastases to induce anti-tumor and pro-osteogenic signaling cascades.

The 1st- and 2nd-generation designs of Propep utilized the signal peptide from tissue plasminogen activator (tPA) (FIG. 2, Tables 1 & 2). tPA, along with peptides derived from secreted alkaline phosphatase (SAP), Gaussia luciferase, human interleukin (IL)-2, serum albumin, and Immunoglobin Kappa light chain (IgG) are among several efficient, well-described, and commercially available signal sequences. More recently, Secrecon (Sec) was identified as the most effective secretion signal in a comparative study of the most commonly used SP sequences. Secrecon is a non-native computationally-designed sequence first described as the ‘ideal human signal sequence.’ While the presence of alanine as a SP adjacent amino acid or linker enhanced the secretion of efficiency of some naturally secreted protein, the absence of any “linker” residues enhanced Sec-mediated section of human IL-25 and human interferon alpha 2a (IFNa2). As interleukins and interferon are key factors in PCa bone metastases survival and proliferation, the enhanced design of the cytokine-based propeptide therapeutic agent incorporated this ideal SP sequence to further improve its expression and secretion in the mouse muscle cell system.

### Example 5: The Tumor Inhibitory Module of Propep

Cytokines in the interleukin (IL)-6 and IL-11 families play critical roles in malignant interactions between tumor and bone cells. In particular, IL-6 and its receptor IL-6Ra are upregulated in mCRPC and play into the ‘vicious cycle’ of tumor:bone malignancy. As a result, therapeutic strategies targeting IL-6 signaling pathway have been extensively explored. However, very few have patients experience lasting clinical benefits due to developing resistance to therapy. Thus, research for IL-6 pathway inhibitors shifted towards antagonist peptides due to their lower production costs, and low immunogenicity. One such agent is the peptide (LSLITRL (SEQ ID NO:5)), a peptide discovered through phage display library screening. Preclinical studies have shown that this peptide specifically binds to IL-6Rα and inhibits IL-6 mediated cancer cell survival and anti-apoptosis signaling cascades. Furthermore, treatment with this antagonist significantly suppressed IL-6 induced angiogenesis and tumor growth in vitro and in vivo. Therefore, this peptide was selected as the anti-tumorigenesis module in the design of Propep (FIG. 1A).

### Example 6: The Pro-Osteogenic Module of Propep

One of the hallmarks of CRPC progression and treatment resistance is presence of bone metastases (mCRPC). It disrupts the balance between osteoblasts (bone formation) and osteoclasts (bone resorption), weakening the structural integrity of the skeleton and thus increasing the risk of bone fractures and morbidity. mCRPC is characterized as osteoblastic in nature, meaning that, in the presence of PCa, it induces aberrant bone formation lacking in proper calcium and mineral deposition. To address this facet of mPCa disease progression, osteostatin (TRSAW (SEQ ID NO:6)) peptide was incorporated in the design of Propep. This peptide is a parathyroid hormone-derived analog that specifically binds the parathyroid hormone type 1 receptor (PTH1R) typically expressed on osteoblasts. PTH-analogs are considered as the most efficacious in building bone, and currently, PHT-14 is the only FDA-approved agent for osteoporosis in the United States. Treatment with PTH analogs induces formation of bone (osteoanabolic effect), followed by an increase in bone resorption. Additionally, PTH analogs expedite fracture healing and the formation of healthy mineralized bone. More recently, Osteostatin has been shown to inhibit oxidative stress and promote differentiation in osteoblastic cells. Therefore, to enable the restoration of bone homeostasis, osteostatin was used as the pro-osteogenic motif in the design of Propep.

### Example 7: The Matrix Metalloproteases Processing Module of Propep

Matrix metalloproteases (MMPs) play critical roles in all hallmarks of tumor development, including promoting tumor cell proliferative signaling, anti-apoptosis, angiogenesis, and activation of invasion and metastases. For instance, MMPs are overexpressed at the interface between tumor and stromal cells, triggering osteolysis and bone metastases. In particular, MMP-2 and MMP-9 are overexpressed and activated at several critical steps of tumor progression and metastases. In designing Propep, this MMP-rich characteristic of cancer bone metastases to process or specifically activate the various therapeutic modules at the site of tumor/bone metastases was utilized. MMP-2/MMP-9 selective cleavage sequences were introduced between each therapeutic module (FIG. 2 and Table 2). Additionally, the design utilizes amino acid residues as linkers adjacent to MMPs cleavage sites. Linkers are often incorporated in the design of recombinant fusion proteins to introduce spatial freedom between domains to construct stable, bioactive proteins. Therefore, linkers were incorporated into the design to stabilize the Propep and maximize accessibility of MMPs cleavage sites (FIG. 2 and Table 2).

### Example 8: Selection of the Most Favorable Propep Model

The predicted secondary structure of the original Propeptide design (1st generation) predominantly displayed unstable structures of random coils (FIGS. 3A-3B). To improve this design, spacers (or linkers) were introduced to the design of second generation of Propep to enhance its structural stability. The second generation of Propep included three α-helices rendering the design more stable and representative of a cytokine-like structure (FIGS. 3C-3D). Propep D was selected for synthesis and the initial development of Propep-based gene therapy. However, this design failed synthesis and QA/QC outsourced to Biomatik Inc. Therefore, 3rd generation Propeptides were developed to enhance their structural stability of Propep and solvent accessibility of matrix metalloproteases cleavage sites, which are critical for Propeptide bioactivity (FIGS. 3E-3H). Propep H was selected for validation and development of Propep-based gene therapy due to the score of its secondary structure prediction making this model likely to be stable and expressed in cells, and due to its high solvent accessibility of MMPs cleavage sites, making them available for matrix metalloproteases processing.

### Example 9: Propep Expression Analysis

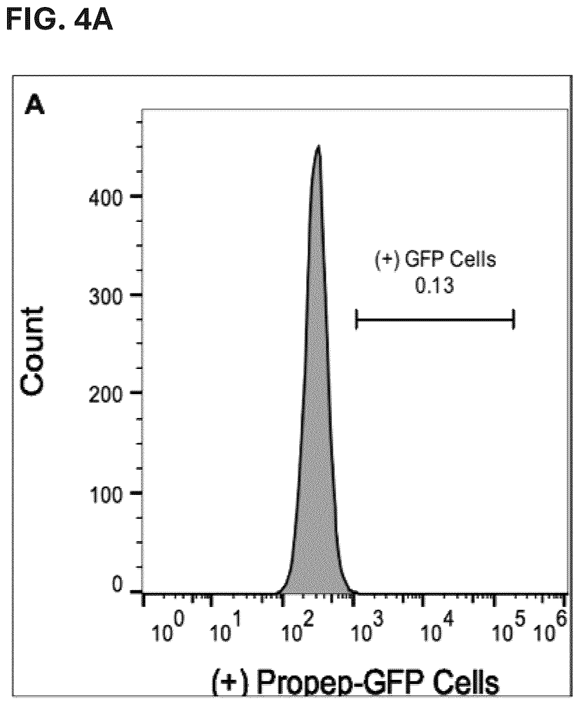

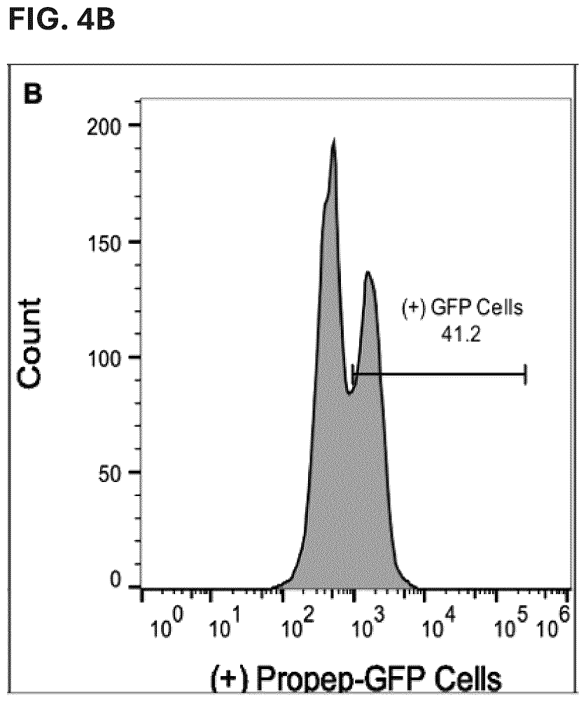

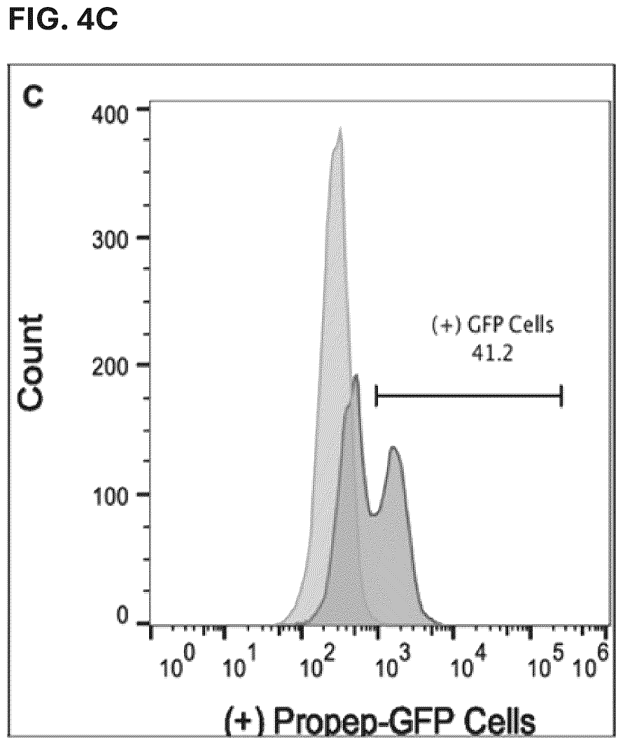

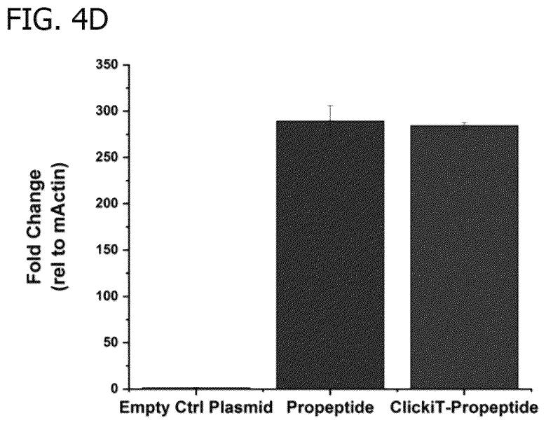

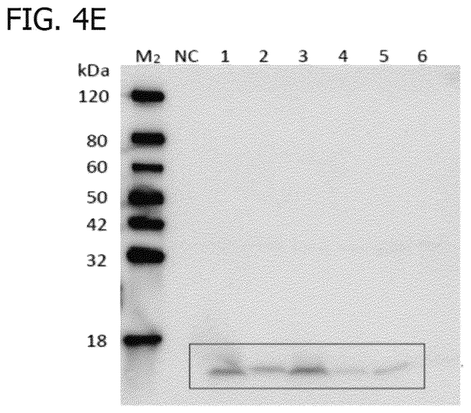

Cell expression of the selected Propep design was validated in three different ways. First, as a GFP fusion, C2C12 cells were transfected with pPropep-GFP and their fluorescence was evaluated using fluorescence activated cell sorting (FACS) (FIGS. 4A-4C). It was found that 41.2% of transfected cells expressed measurable quantities of Propep-GFP. Second, the gene expression levels of label-free Propep and ClickiT-Propep plasmid constructs following transfection in C2C12 were determined. The gene expression levels of both label-free constructs were 300-fold greater than the housekeeping gene mouse b-actin (FIG. 4D). Lastly, His-tagged Propep was expressed in E. coli cells and the expression was assessed via Western blot (FIG. 4E).

### Example 10: Selective Processing of Propep with MMP-9

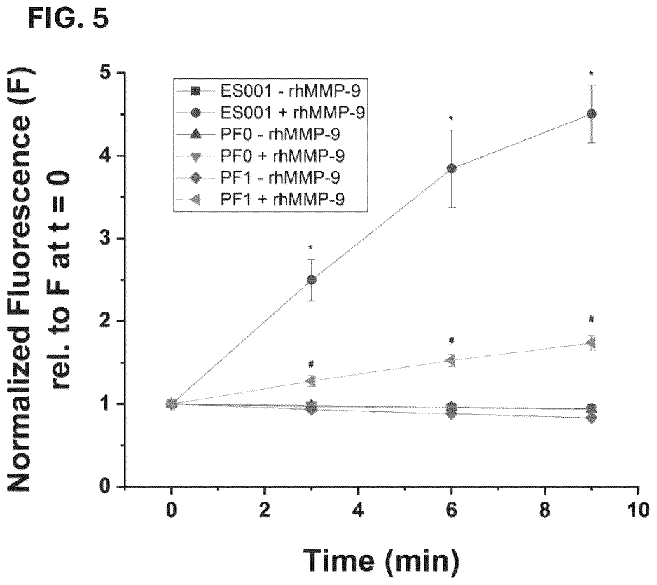

To validate the selective cleavage of Propep, custom fluorescently quenched peptides PF0 were designed not to include the MMP-9 cleavage site, whereas PF1 was designed to include one MMP-9 cleavage site. A commercially available fluorescently quenched peptide (ES001) containing an MMP-9 cleavage site was used as a positive control. All three peptides were assayed in the presence or absence of rhMMP-9 in either assay buffer (TCNB) or prostate cancer cells conditioned media (CM). Peptides containing an MMP-9 cleavage site (ES001 and PF1) exhibited a statistically significant increase in fluorescence following the initiation of the reaction with rhMMP-9 in TCBN relative to controls (FIG. 5). This increase in fluorescence was not observed in the absence of rhMMP-9. As expected, peptide FPO did not exhibit any changes in fluorescence either in the presence or absence of rhMMP-9 (FIG. 5).

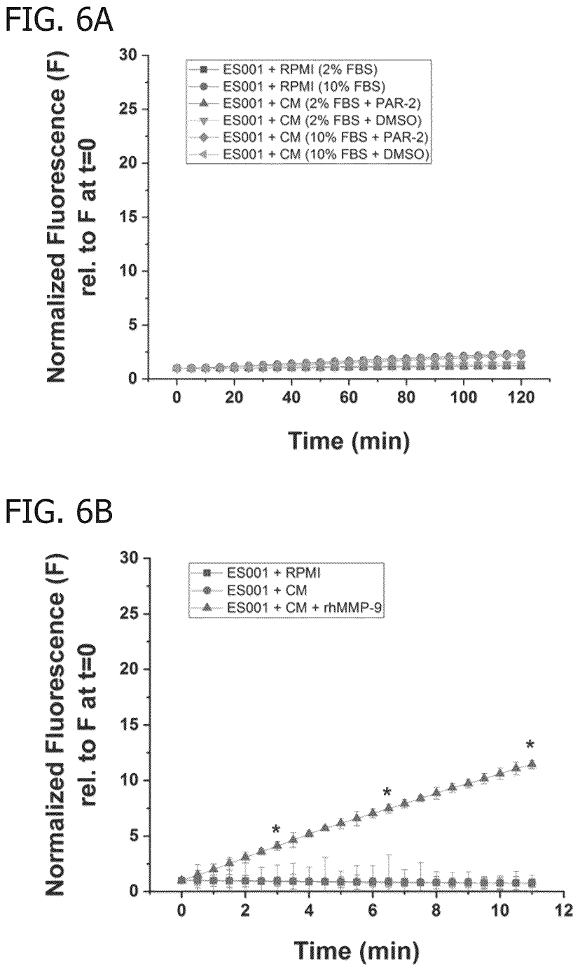

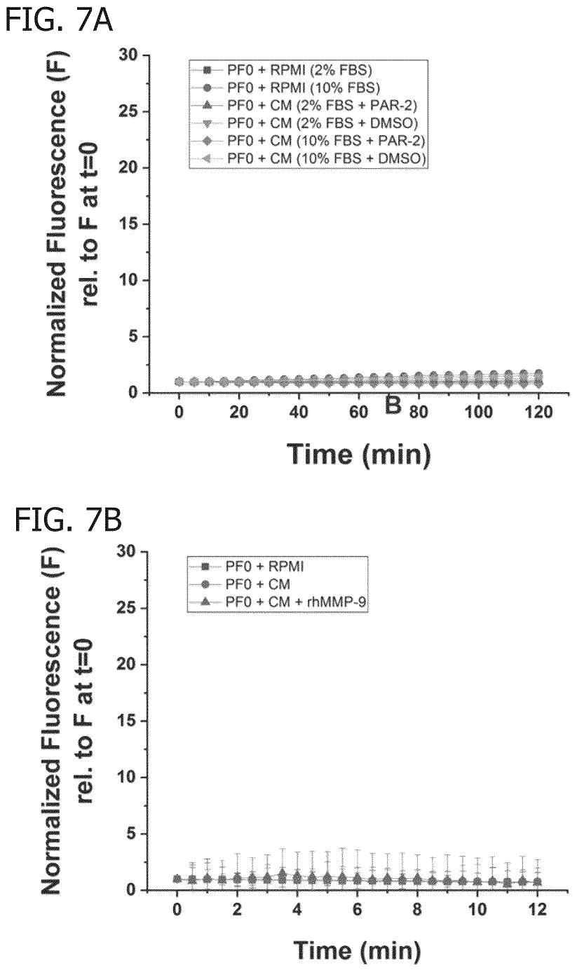

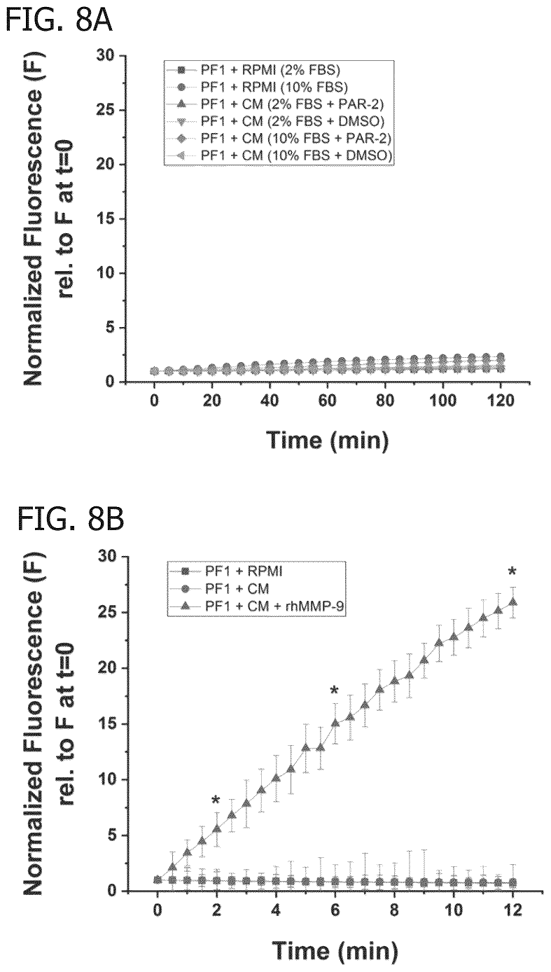

The selective processing of Propep in cancer cells conditioned media from C4-2B cells was also validated. Peptides ES001 and PF1, which include the cleavage site for MMP-9, exhibited a significant increase in fluorescence following assay initiation with rhMMP-9 (FIGS. 6A-6B and 8A-8B). No changes in fluorescence intensities were observed in RPMI media or CM. PF0 peptide, which excludes the cleavage site for MMP-9, exhibited no changes in fluorescence in any medium (RPMI, CM, or CM+rhMMP-9) (FIGS. 7A-7B).

### Example 11: Selective Targeting of Propep in C4-2B Cancer Calls

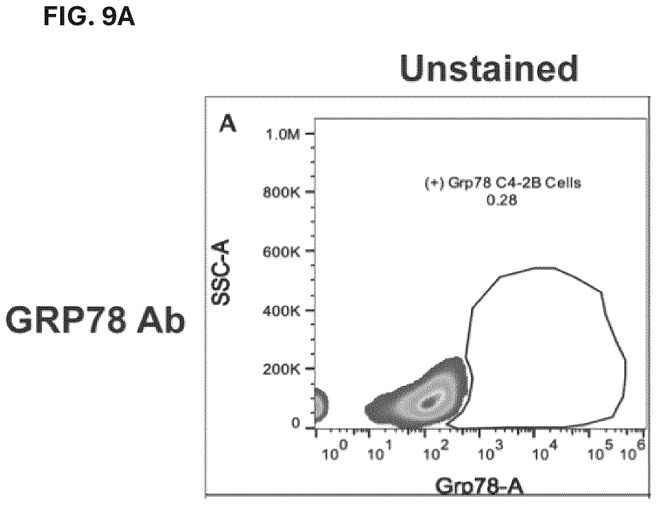

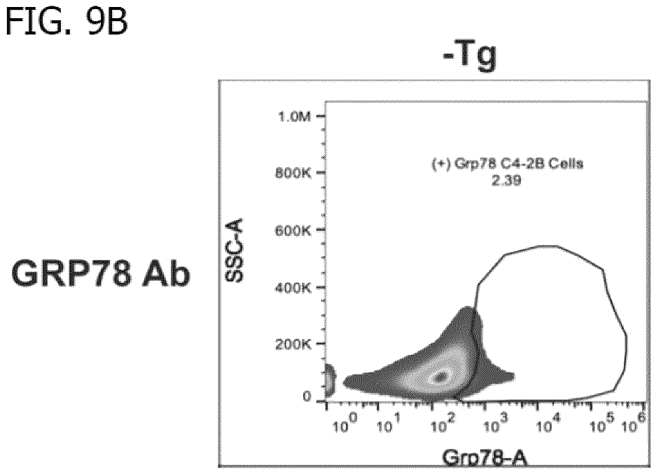

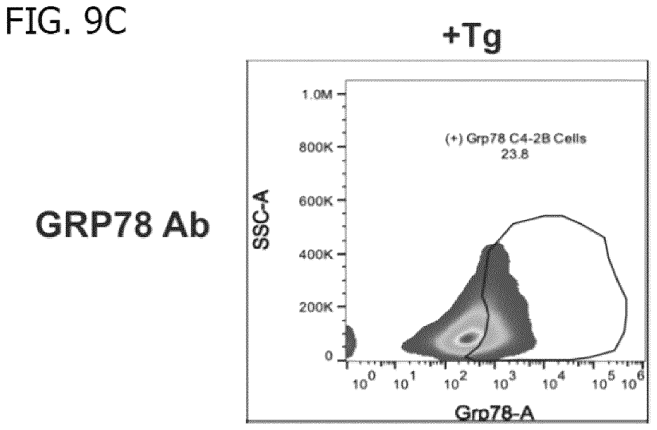

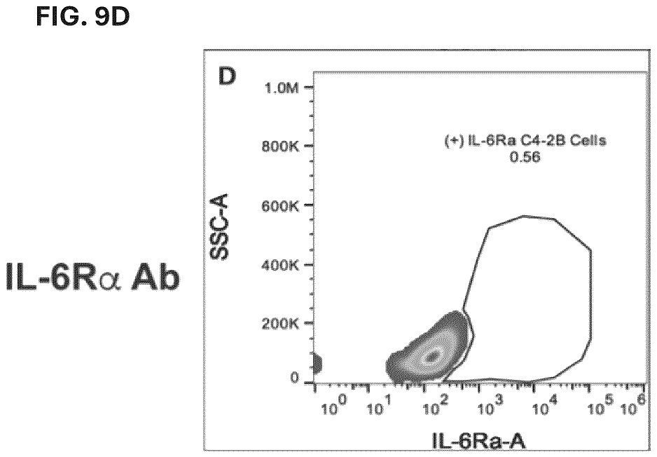

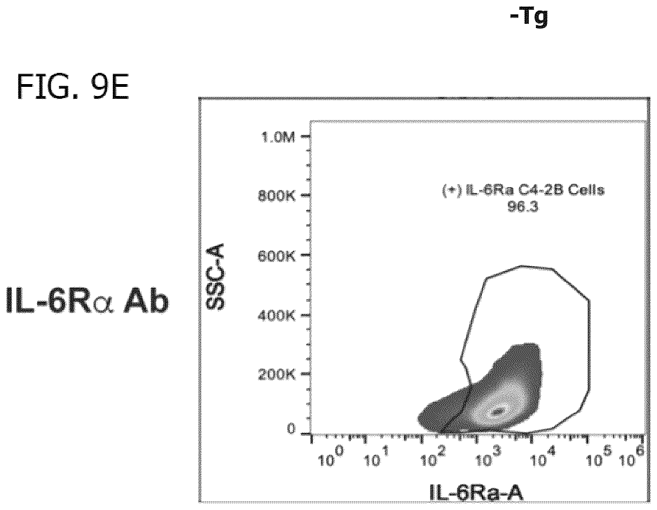

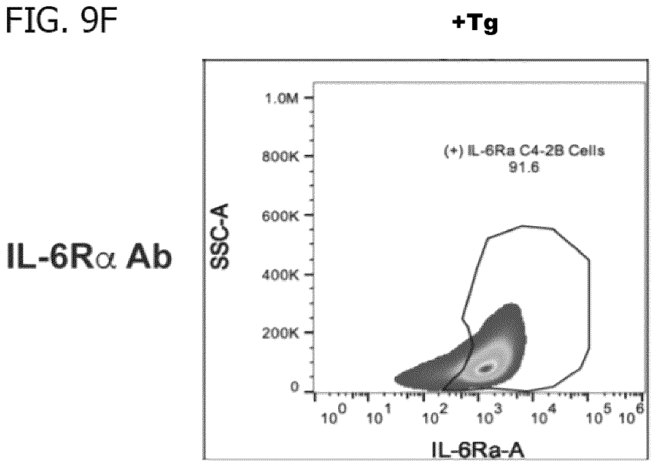

The glucose response protein-78 (GRP78) is the receptor target of Propep. FACS was utilized to evaluate the endogenous expression of GRP78 on the surface of C4-2B cells. Only 2.39% of cells displayed the target receptor on their cell surface (FIG. 9B). Following treatment with Thapsigargin (Tg) to promote ER stress, 23.8% of C4-2B cells displayed GRP78 on their cell surface (FIG. 9C). Treatment with Tg did not significantly reduce endogenous IL-6Ra as a control on the surface of C4-2B cells, and this is expected since this receptor is not known to respond to ER stress (FIGS. 9E-9F). Over 90% of C4-2B cells expressed IL-6Ra in the presence or absence of Tg treatment, as expected.

The targeting ability of Propep was evaluated using a FITC-labeled peptide containing the targeting module, pepG. Tg-treated C4-2B were incubated with 0-100 uM of FITC-pepG, resulting in a dose-response curve with a half maxima effective concentration of 40 uM (FIG. 10).

### Example 12: STAT-1/ -3 Activation in C4-2B Cancer Cells

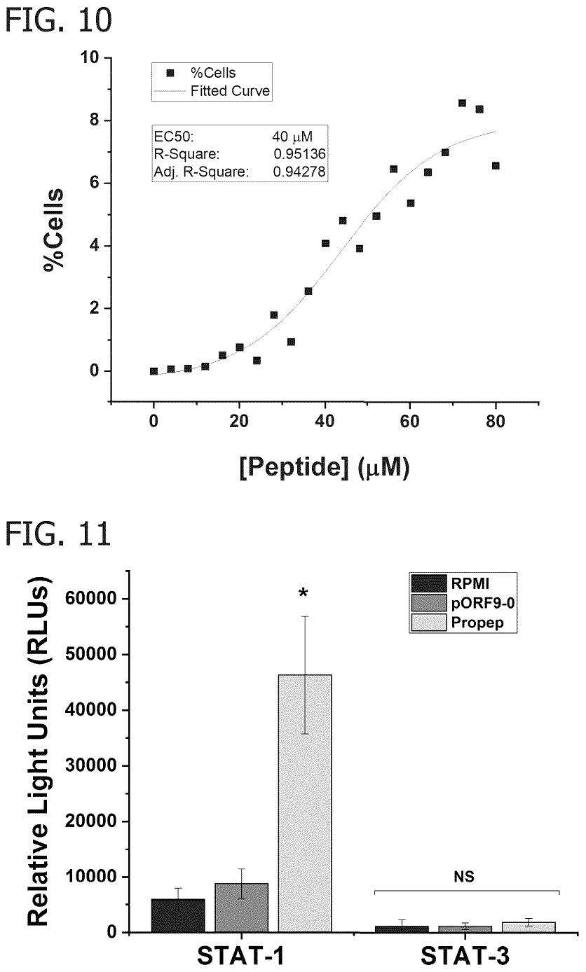

A functional assay was conducted to estimate the anti-tumorigenic and pro-apoptotic potential of the targeted Propeptide therapeutic through STAT signaling shifts (FIG. 11). Treatment with Propeptide significantly upregulated STAT-1 expression in C4-2B cells transfected with a STAT-1 luciferase reporter (pGAS/ISRE-Luc). Oncogenic STAT-3 signaling was only slightly upregulated due to treatment with Propep, but this difference was statistically insignificant compared to controls. This is expected due to overlapping signaling factors, and homo- or hetero-dimerization of the signal transduction subunit with IL-6 family receptors. However, the treatment with Propep pivots primarily towards STAT-1 activation.

### DISCUSSION

In the above Examples, the design and activity validation of a theoretical cytokine mimic were explored. While earlier attempts to chemically synthesize first and second generation Propep failed to produce a structurally stable final product, there was a successful transition from theory to application by relying on gene delivery and expression in cell systems. Gene and protein expression analysis demonstrated that the selected Propeptide model resulted in a structurally stable molecule detectable by RT-qPCR, western blot, and FACS analysis.

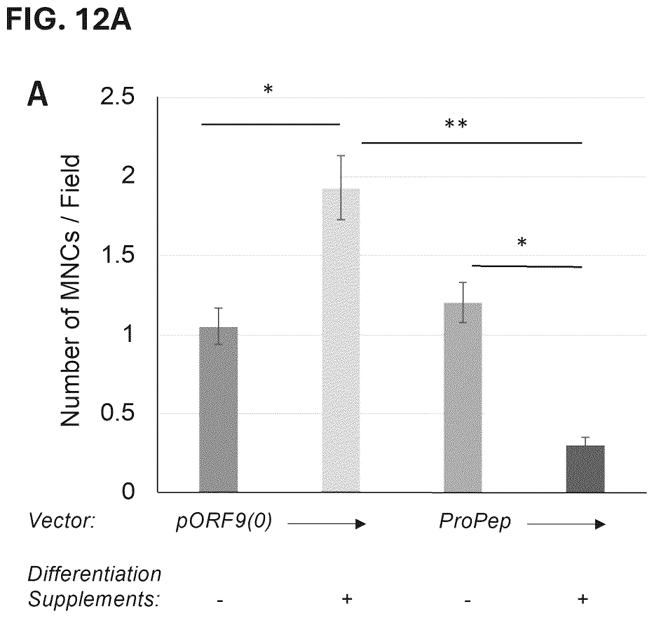

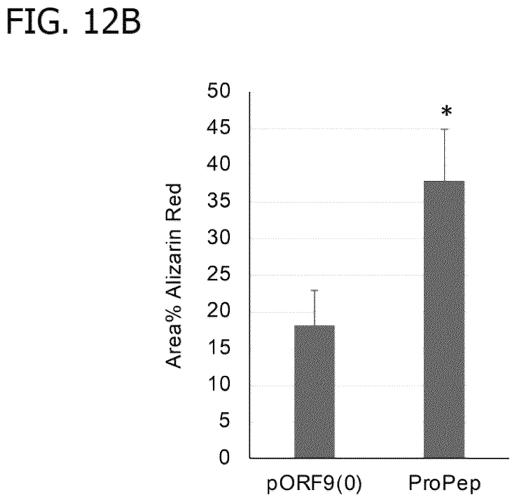

The 3rd generation Propep design differs from the 1st- and 2nd-generation Propep in its secretion (Secrecon) and targeting modules (pepG). The therapeutic modules of Propep remained the same: the anti-tumorigenic module (pepL), and the pro-osteogenic module (pepB). The anti-tumorigenic activity of pepL has been previously reported. More recently, it has been reported that pepL enhanced IL-27 ani-tumor activity in vivo relative to non-targeted IL-27 and empty plasmid control by reducing overall prostate tumor volume (TC2Ras C57/BL6 cells) over time. Propep may have pro-osteogenic activity through increasing OB differentiation and reducing OC differentiation (FIGS. 12A-12B).

The presence of matrix metalloproteases cleavage sites is a key feature of Propep design, to allow for the therapeutic modules of Propep to be released at the site of metastases and individually bind their target receptors. To test this feature, two fluorescently quenched peptides (PF0 and PF1) composed of pepL and pepG were custom ordered. PF0 did not contain an MMP cleavage site, while PF1 contained a single MMP cleavage site. ES001, a commercially available MMP substrate composed only of a single peptide sequence for one MMP cleavage site. Both ES001 and PF1 exhibited an increase in relative fluorescence units following the addition of rhMM-9, which was not observed in the absence of rhMMP-9 from the reaction. PF0 did not exhibit any changes in relative fluorescence units with either the presence or the absence of rhMMP-9. These results were also replicated in C4-2B conditioned media, confirming that Propeptide ‘activation’ can be achieved in cell culture. Furthermore, Propeptide activation in conditioned media induced significant activation of the desired anti-tumorigenic STAT-1 reporter in C4-2B prostate cancer cells. Also promising were results that showed that Propeptide activation in conditioned media did not promote the pro-tumorigenic signaling pathway STAT-3 in C4-2B prostate cancer cells.

The targeting modality of Propep in C4-2B cells by utilizing a FITC-label Propep fragment composed only of pepG for FACS analysis has also been evaluated. Despite reports of GRP78 (the target receptor for pepG) being overexpressed and displayed on the surface of prostate cancer cells, the initial GRP78 C4-2B characterization using labeled antibodies showed that only 2.39% of the cells displayed GRP78. Therefore, this experiment was repeated to include C4-2B cells that have been treated with thapsigargin (Tg) to induce ER stress and the translocation of GRP78 to the cell surface. Approximately 20-30% of Tg-treated C4-2B cells displayed measurable amounts of GRP78 on their cell surface. No changes in IL-6Ra expression were observed following Tg-treatment, as expected, since this receptor is not known to be displayed in response to cellular stress. Taken together, these results provided a system in which both the homing receptor (GRP78) and the therapeutic target receptor (IL-6Ra) were available in detectable quantities to evaluate the cytokine-based therapeutic, Propep. The dose response analysis of Tg-treated C4-2B were incubated with 0-100 uM FITC labeled pepG showed that pepG has an EC50 of 40 uM. This EC50 value appears to have a lower potential for targeting than originally expected. However, several studies have shown in vivo that GRP78 can be effectively targetable with peptides such as pepG (SNTRVAP (SEQ ID NO:2). For example, GRP78 peptide ligands (WIFPWIQL (SEQ ID NO:20) and WDLAWMFRLPVG (SEQ ID NO:21)) have been shown to specifically target, bind, and undergo internalization in DU145 cancer cells. Importantly, in vivo studies demonstrated that GRP78 binding peptides specifically home to tumors following systemic treatment with little to no accumulation in off-target organs. More recent work reported that GRP78 peptide ligand (pepG; SNTRVAP (SEQ ID NO:2)) specifically targets a model of CRPC osteogenic prostate cancer in vivo. Even though this tumor homing peptide has not been extensively explored as a targeting module in gene therapy approaches, and resulted in a high EC50 in C4-2B cells, numerous studies have effectively utilized it for targeting. More recently, SNTRVAP (SEQ ID NO:2)-displaying adeno-associated virus/phage (AAVP) has been effectively utilized to deliver human Herpes simplex virus thymidine kinase type-1, which serves as both a noninvasive imaging reporter and a suicide transgene, in xenograft-bearing mice. Based on literature, it is thought that this module should remain the most potentially effective in delivering the Propeptide to the site of bone metastases in an in vivo system, where often the therapeutics can display their full potential relative to cell culture systems.

### Example 13: Rheumatoid-Arthritis

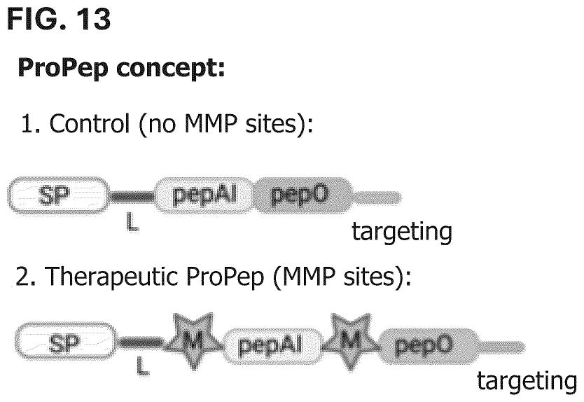

The therapeutic ‘propeptides’ (ProPep) can also be given via gene delivery to treat inflammatory and bone lesions associated with rheumatoid arthritis, for example. ProPep structures can contain dual therapeutic domains composed of an anti-inflammatory and an osteogenesis-promoting (or anti-resorptive) module (FIG. 13). They can be designed with or without cleavage sites for processing by matrix metalloproteases (MMP) upregulated at RA lesions.

The Propep will be produced from a skeletal muscle cell ‘depot’ and will be targeted for accumulation at affected joints through targeting to IL-6Rα through pepL (LSLITRL) (SEQ ID NO: 5) sequence, which is upregulated in inflammatory and bone cells in the joint. Accumulation of ProPep can also be enhanced at joints via an albumin-binding nanobody.

1. A propeptide comprising a secretion peptide, a pro-osteogenic peptide, an anti-tumorigenic peptide, and a tumor targeting peptide,
wherein the secretion peptide comprises Secrecon comprising amino acid sequence MWWRLWWLLLLLLLLWPMVWA (SEQ ID NO: 1);
wherein the tumor targeting peptide comprises pepG comprising the GRP78 targeting peptide SNTRVAP (SEQ ID NO:2);
wherein each peptide is separated by a matrix metalloprotease (MMP) cleavage site comprising amino acid sequence GPLGMLSQ (SEQ ID NO:3) or a prostate specific antigen (PSA) cleavage site comprising amino acid sequence RSSYYSL (SEQ ID NO:4).

2. The propeptide of claim 1, wherein the anti-tumorigenic peptide comprises pepL comprising amino acid sequence LSLITRL (SEQ ID NO:5).

3. The propeptide of claim 1, wherein the pro-osteogenic module comprises pepB comprising osteostatin peptide TRSAW (SEQ ID NO:6).

4. The propeptide of m claim 1, wherein the propeptide further comprises a reporter.

5. The propeptide of claim 4, wherein the reporter comprises ClickiT or GFP.

6. The propeptide of claim 1, wherein the propeptide further comprises linkers or spacer peptides that enhance the solvent accessibility of the MMP or PSA cleavage sites.

7. (canceled)

8. The propeptide of claim 1, wherein the propeptide comprises the amino acid sequence of SecGRSG-GRP78 or ScGRSG1.3-GPR78.

9. (canceled)

10. The propeptide of claim 1, wherein the propeptide further comprises amino acid sequence PAS.

11.-13. (canceled)

14. A method of treating prostate cancer bone metastases in a subject in need thereof, the method comprising
administering a gene therapy comprising a DNA sequence encoding the propeptide of claim 1 to a muscle near the metastases, wherein the propeptide is produced and secreted from muscle cells in the muscle;
wherein the propeptide is targeted to the metastases by the tumor targeting peptide;
wherein the propeptide is cleaved into the secretion peptide, the pro-osteogenic peptide, the anti-tumorigenic peptide, and the tumor targeting peptide by MMPs found at the metastases;
wherein the pro-osteogenic peptide restores bone homeostasis; and
wherein the anti-tumorigenic peptide suppresses growth of the metastases.

15. The method of claim 14, wherein the prostate cancer is androgen-independent prostate cancer or metastatic castration resistant prostate cancer.

16. The method of claim 14, wherein administering the gene therapy comprises administration via adeno-associated virus/phage or plasmid.

17. The method of claim 16, wherein the adeno-associated virus/phage is AAV or Ad5.

18. A propeptide comprising a secretion peptide, a pro-osteogenic peptide, an anti-inflammatory peptide, and a joint targeting peptide,
wherein the secretion peptide comprises Secrecon comprising amino acid sequence MWWRLWWLLLLLLLLWPMVWA (SEQ ID NO: 1);
wherein the joint targeting peptide comprises pepL comprising the IL-6Ra targeting peptide LSLITRL (SEQ ID NO:5);
wherein each peptide is separated by a matrix metalloprotease (MMP) cleavage site comprising amino acid sequence GPLGMLSQ (SEQ ID NO:3).

19. The propeptide of claim 18, wherein the pro-osteogenic module comprises pepB comprising osteostatin peptide TRSAW (SEQ ID NO:6).

20. The propeptide of claim 18, wherein the propeptide further comprises a reporter.

21. (canceled)

22. The propeptide of claim 18, wherein the propeptide further comprises linkers or spacer peptides that enhance the solvent accessibility of the MMP cleavage sites.

23. (canceled)

24. The propeptide of claim 18, wherein the propeptide further comprises amino acid sequence PAS.

25.-27. (canceled)

28. A method of treating rheumatoid arthritis in a subject in need thereof, the method comprising administering a gene therapy comprising a DNA sequence encoding the propeptide of claim 18 to a muscle near an arthritic joint in the subject, wherein the propeptide is produced and secreted from muscle cells in the muscle;
wherein the propeptide is targeted to the arthritic joint by the joint targeting peptide;
wherein the propeptide is cleaved into the secretion peptide, the pro-osteogenic peptide, the anti-inflammatory peptide, and the joint targeting peptide by MMPs found at the joint;
wherein the pro-osteogenic peptide restores bone homeostasis; and
wherein the anti-inflammatory peptide suppresses inflammation in the joint.

29. The method of claim 28, wherein administering the propeptide comprises administration via adeno-associated virus/phage or plasmid.

30. The method of claim 29, wherein the adeno-associated virus/phage is AAV or Ad5.
